(19) United States
(12) Patent Application Publication
(10) Pub. No.: US 2010/0204249 A1
(43) Pub. Date: Aug. 12, 2010

(54) SECRETORY PHOSPHOLIPASE A2 (SPLA2) INHIBITOR AND NIACIN DRUG COMPOSITIONS AND METHODS FOR TREATING CARDIOVASCULAR DISEASE AND DYSLIPIDEMIA

(76) Inventors: Colin HISLOP, Menlo Park, CA (US); Joaquim Trias, Millbrae, CA (US); Debra Odink, Oakland, CA (US); Bernadine Fraser, Sunnyvale, CA (US); Scott Chadwick, Redwood City, CA (US)

Correspondence Address: PERKINS COIE LLP, POST OFFICE BOX 1208, SEATTLE, WA, 98111-1208, US

(21) Appl. No.: 12/684,757

(22) Filed: Jan. 8, 2010

Related U.S. Application Data
(60) Provisional application No. 61/143,373, filed on Jan. 8, 2009.

(51) Int. Cl. A61K 31/455; A61K 31/4965; A61K 31/52; A61K 31/465; A61P 9/10; A61P 3/06; A61P 9/00

(52) U.S. Cl. 514/263.22; 514/356; 514/255.06; 514/355

(57) ABSTRACT

Niacin drugs are frequently utilized as a therapeutic to treat CVD, increase HDL levels, and/or decrease TG levels. As disclosed herein, it has been found that administration of one or more sPLA₂ inhibitors in combination with one or more niacin drugs unexpectedly results in a synergistic increase in HDL levels and a synergistic decrease in TG levels. Therefore, compositions, methods, and kits are provided for treating CVD, increasing HDL levels, decreasing TG levels, and improving HDL/LDL ratios.

### RELATED APPLICATIONS

The present application claims the benefit of U.S. Provisional Application No. 61/143,373, filed Jan. 8, 2009, the disclosure of which is incorporated by reference herein in its entirety, including drawings.

### BACKGROUND

In 2004, it was estimated that over 75 million Americans had one or more forms of cardiovascular disease (CVD), including coronary heart disease (CHD) and coronary artery disease (CAD). Among the most popular and effective therapeutic options for treating CVD are statins and niacin drugs. Administration of statins has been shown to decrease LDL and triglyceride (TG) levels, while administration of niacin drugs has been shown to decrease TG levels and increase HDL levels. Although these therapies have succeeded in treating CVD to a limited extent, neither has been completely effective. Therefore, there is a need in the art for improved methods of treating CVD.

### SUMMARY

In certain embodiments, compositions are provided comprising one or more sPLA₂ inhibitors and one or more niacin drugs. In certain of these embodiments, the one or more sPLA₂ inhibitors comprise 3-(2-Amino-1,2-dioxoethyl)-2-ethyl-1-(phenylmethyl)-1H-indol-4-yl)oxy)acetic acid (A-001) or a pharmaceutically acceptable prodrug, salt, solvate, polymorph, or co-crystal thereof. In certain of these embodiments, the prodrug of A-001 is a C₁-C₆ alkyl ester prodrug, an acyloxyalkyl ester prodrug, or an alkyloxycarbonyloxyalkyl ester prodrug, and in certain of these embodiments the prodrug is [[3-(2-Amino-1,2-dioxoethyl)-2-ethyl-1-(phenylmethyl)-1H-indol-4-yl]oxy]acetic acid methyl ester (A-002). In certain embodiments, the niacin drug is niacin, nicotinic acid, acipimox, immediate release niacin, extended release niacin, extended release niacin in combination with lovastatin, extended release niacin in combination with simvastatin, extended release niacin in combination with laropiprant, xanthinol niacinate, 1-methylnicotinamide, and 1-methyl-N′-hydroxymethylnicotinamide. In certain embodiments, the composition further comprises one or more statins. In certain embodiments, the composition further comprises one or more pharmaceutically acceptable carriers.

In certain embodiments, methods are provided for increasing HDL levels in a subject by administering a therapeutically effective amount of one or more sPLA₂ inhibitors and a therapeutically effective amount of one or more niacin drugs. In certain of these embodiments, the one or more sPLA₂ inhibitors comprise A-001 or a pharmaceutically acceptable prodrug, salt, solvate, polymorph, or co-crystal thereof. In certain of these embodiments, the prodrug of A-001 is a C₁-C₆ alkyl ester prodrug, an acyloxyalkyl ester prodrug, or an alkyloxycarbonyloxyalkyl ester prodrug, and in certain of these embodiments the prodrug is A-002. In certain embodiments, the niacin drug is niacin, nicotinic acid, acipimox, immediate release niacin, extended release niacin, extended release niacin in combination with lovastatin, extended release niacin in combination with simvastatin, extended release niacin in combination with laropiprant, xanthinol niacinate, 1-methylnicotinamide, and 1-methyl-N′-hydroxymethylnicotinamide. In certain embodiments, one or more statins are also administered to the subject.

In certain embodiments, methods are provided for decreasing TG levels in a subject by administering a therapeutically effective amount of one or more sPLA₂ inhibitors and a therapeutically effective amount of one or more niacin drugs. In certain of these embodiments, the one or more sPLA₂ inhibitors comprise A-001 or a pharmaceutically acceptable prodrug, salt, solvate, polymorph, or co-crystal thereof. In certain of these embodiments, the prodrug of A-001 is a C₁-C₆ alkyl ester prodrug, an acyloxyalkyl ester prodrug, or an alkyloxycarbonyloxyalkyl ester prodrug, and in certain of these embodiments the prodrug is A-002. In certain embodiments, the niacin drug is niacin, nicotinic acid, acipimox, immediate release niacin, extended release niacin, extended release niacin in combination with lovastatin, extended release niacin in combination with simvastatin, extended release niacin in combination with laropiprant, xanthinol niacinate, 1-methylnicotinamide, and 1-methyl-N′-hydroxymethylnicotinamide. In certain embodiments, one or more statins are also administered to the subject.

In certain embodiments, methods are provided for increasing HDL/LDL ratio in a subject by administering a therapeutically effective amount of one or more sPLA₂ inhibitors and a therapeutically effective amount of one or more niacin drugs. In certain of these embodiments, the one or more sPLA₂ inhibitors comprise A-001 or a pharmaceutically acceptable prodrug, salt, solvate, polymorph, or co-crystal thereof. In certain of these embodiments, the prodrug of A-001 is a C₁-C₆ alkyl ester prodrug, an acyloxyalkyl ester prodrug, or an alkyloxycarbonyloxyalkyl ester prodrug, and in certain of these embodiments the prodrug is A-002. In certain embodiments, the niacin drug is niacin, nicotinic acid, acipimox, immediate release niacin, extended release niacin, extended release niacin in combination with lovastatin, extended release niacin in combination with simvastatin, extended release niacin in combination with laropiprant, xanthinol niacinate, 1-methylnicotinamide, and 1-methyl-N′-hydroxymethylnicotinamide. In certain embodiments, one or more statins are also administered to the subject. In certain embodiments, the increase in HDL/LDL ratio is accomplished at least in part by increasing HDL levels.

In certain embodiments, methods are provided for treating CVD or a condition associated with CVD in a subject in need thereof by administering a therapeutically effective amount of a therapeutically effective amount of one or more sPLA₂ inhibitors and a therapeutically effective amount of one or more niacin drugs. In certain of these embodiments, the one or more sPLA₂ inhibitors comprise A-001 or a pharmaceutically acceptable prodrug, salt, solvate, polymorph, or co-crystal thereof. In certain of these embodiments, the prodrug of A-001 is a C₁-C₆ alkyl ester prodrug, an acyloxyalkyl ester prodrug, or an alkyloxycarbonyloxyalkyl ester prodrug, and in certain of these embodiments the prodrug is A-002. In certain embodiments, the niacin drug is niacin, nicotinic acid, acipimox, immediate release niacin, extended release niacin, extended release niacin in combination with lovastatin, extended release niacin in combination with simvastatin, extended release niacin in combination with laropiprant, xanthinol niacinate, 1-methylnicotinamide, and 1-methyl-N′-hydroxymethylnicotinamide. In certain embodiments, one or more statins are also administered to the subject. In certain embodiments, the CVD being treated is CAD, CHD, or a condition associated with CAD or CHD, and in certain of these embodiments the condition is ACS and/or dyslipidemia.

In certain embodiments, methods are provided for increasing the effectiveness of niacin drug administration in a subject by administering a therapeutically effective amount of one or more sPLA₂ inhibitors in conjunction with the niacin drug. In certain of these embodiments, the one or more sPLA₂ inhibitors comprise A-001 or a pharmaceutically acceptable prodrug, salt, solvate, polymorph, or co-crystal thereof. In certain of these embodiments, the prodrug of A-001 is a C₁-C₆ alkyl ester prodrug, an acyloxyalkyl ester prodrug, or an alkyloxycarbonyloxyalkyl ester prodrug, and in certain of these embodiments the prodrug is A-002. In certain embodiments, the niacin drug is niacin, nicotinic acid, acipimox, immediate release niacin, extended release niacin, extended release niacin in combination with lovastatin, extended release niacin in combination with simvastatin, extended release niacin in combination with laropiprant, xanthinol niacinate, 1-methylnicotinamide, and 1-methyl-N′-hydroxymethylnicotinamide. In certain embodiments, administration of one or more sPLA₂ inhibitors in conjunction with the niacin drug causes a greater increase in HDL and/or decrease in TG than administration of the niacin drug by itself. In certain embodiments, one or more statins are also administered to the subject.

In certain embodiments, kits are provided that comprise one or more sPLA₂ inhibitors and one or more niacin drugs. In certain embodiments, these kits further comprise one or more statins. In certain embodiments, the kits further comprise instructions for usage.

In certain embodiments, the use of one or more sPLA2 inhibitors and one or more niacin drugs for producing a medicament for lowering TG levels, increasing HDL levels, increasing HDL/LDL ratio, and/or treating CVD or a condition associated with CVD is provided.

### DETAILED DESCRIPTION

The following description of the invention is merely intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

### ABBREVIATIONS

A-001, 3-(2-Amino-1,2-dioxoethyl)-2-ethyl-1-(phenylmethyl)-1H-indol-4-yl)oxy)acetic acid; A-002, [[3-(2-Amino-1,2-dioxoethyl)-2-ethyl-1-(phenylmethyl)-1H-indol-4-yl]oxy]acetic acid methyl ester; ACS, acute coronary syndrome; CAD, coronary artery disease; CHD, coronary heart disease; CVD, cardiovascular disease; ERN, extended release niacin; HDL, high density lipoprotein; hs-CRP, high-sensitivity C-reactive protein; LDL, low density lipoprotein; NAc, nicotinic acid; NCEP, National Cholesterol Education Program; NHLBI, National Heart Lung and Blood Institute; sPLA₂, secretory phospholipase A₂; TG, triglyceride; VLDL, very low density lipoprotein.

The terms “treat,” “treating,” or “treatment” as used herein with regard to a condition refers to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, lessening the severity of the condition, changing the levels of one or more markers associated with the condition, or some combination thereof. For example, with regard to CVD, “treatment” may refer to a decrease in triglyceride levels, an increase in HDL levels, or some combination thereof.

The term “subject” as used herein refers to any mammal, preferably a human.

A “subject in need thereof” refers to a subject currently diagnosed with CVD or exhibiting one or more conditions associated with CVD, a subject who has been diagnosed with or exhibited one or more conditions associated with CVD in the past, or a subject who has been deemed at risk of developing CVD or one or more conditions associated with CVD in the future due to hereditary or environmental factors such as hypertension, smoking, insulin resistance, infection, or inflammation. In certain of these embodiments, the CVD is CHD, CAD, and/or a condition associated with CHD or CAD, and in certain of these embodiments the subject has been diagnosed with acute coronary syndrome (ACS) or deemed at risk for developing ACS. In certain embodiments, a “subject in need thereof” refers to a subject currently exhibiting elevated TG levels and/or low HDL levels or HDL/LDL ratio, a subject who has exhibited elevated TG levels and/or low HDL levels or HDL/LDL ratio in the past, or a subject who has been deemed at risk for developing elevated TG levels and/or low HDL levels or HDL/LDL ratio.

The term “TG level” as used herein may refer to blood TG level, serum TG level, plasma TG level, or TG level from another biological fluid. The term “elevated TG level” as used herein refers to a TG level that is above an accepted normal threshold level, such as the one promulgated by the National Heart Lung and Blood Institute (NHLBI) National Cholesterol Education Program (NCEP). The accepted normal threshold TG level of a particular subject may vary based on factors such as sex or age, or based on the presence of risk factors such as a prior personal or family history of CVD. In certain embodiments, a subject is deemed to have elevated TG levels if the subject exhibits TG levels of 150 mg/dl or higher. In certain other embodiments, a subject is deemed to have elevated TG levels if the subject exhibits TG levels of 175 mg/dl or higher, in other embodiments 200 mg/dl or higher, in other embodiments 300 mg/dl or higher, in other embodiments 400 mg/dl or higher, and in still other embodiments 500 mg/dl or higher.

The term “HDL level” as used herein may refer to blood HDL level, serum HDL level, plasma HDL level, or HDL level from another biological fluid. The terms “low HDL level” and “low HDL/LDL ratio” as used herein refers to an HDL level or HDL/LDL ratio that is below an accepted normal threshold level, such as the one promulgated by the NHLBI NCEP. The accepted normal threshold HDL level of a particular subject may vary based on factors such as sex or age, or based on the presence of risk factors such as a prior personal or family history of CVD. In certain embodiments, a subject is deemed to have low HDL levels if the subject exhibits HDL levels of less than 60 mg/dl. In certain other embodiments, a subject is deemed to have low HDL levels if the subject exhibits HDL levels of 50 mg/dl or lower, and in other embodiments 40 mg/dl or lower. In certain embodiments, a subject is deemed to have a low HDL/LDL ratio if the subject exhibits an HDL/LDL ratio below 0.25. In certain other embodiments, a subject is deemed to have a low HDL/LDL ratio if the subject exhibits an HDL/LDL ratio of 0.15 or lower, and in other embodiments 0.10 or lower.

An improvement in HDL/LDL ratio as used herein refers to any increase in the ratio of HDL levels to LDL levels, and may be accomplished by increasing HDL levels, decreasing LDL levels, or some combination thereof.

“Cardiovascular disease” or “CVD” as used herein includes, for example, atherosclerosis, including coronary artery atherosclerosis and carotid artery atherosclerosis, CAD, CHD, conditions associated with CAD and CHD, cerebrovascular disease and conditions associated with cerebrovascular disease, peripheral vascular disease and conditions associated with peripheral vascular disease, aneurysm, vasculitis, venous thrombosis, diabetes mellitus, and metabolic syndrome.

“Conditions associated with CVD” as used herein include, for example, dyslipidemia and hypertension. Dyslipidemia refers to any disruption in lipid levels, such as for example hyperlipidemia (elevated lipid levels), hypercholesterolemia (elevated cholesterol levels), hypertriglyceridemia (elevated TG levels), hypo-HDL-emia (low HDL levels), elevated glucose levels, and low HDL/LDL ratio.

“Conditions associated with CAD and CHD” as used herein include, for example, ACS, which in turn includes unstable angina (UA), non-ST-segment elevation myocardial infarction (NSTEMI), and ST-segment elevation myocardial infarction (STEMI).

“Conditions associated with cerebrovascular disease” as used herein include, for example, transient ischemic attack (TIA) and stroke.

“Conditions associated with peripheral vascular disease” as used herein include, for example, claudication.

“Statin” as used herein refers to any compound that inhibits HMG-CoA reductase, an enzyme that catalyzes the conversion of HMG-CoA to mevalonate.

“sPLA₂ inhibitor” as used herein refers to any compound or prodrug thereof that inhibits the activity of sPLA₂.

A “reduction” or “decrease” in the level of a particular lipid or other biomarker may refer to either a reduction versus baseline or a reduction versus placebo. For example, administration of an sPLA₂ inhibitor in conjunction with one or more niacin drugs may reduce TG levels by dropping TG levels below a previously determined baseline level. Alternatively, administration of an sPLA₂ inhibitor in conjunction with one or more niacin drugs may reduce TG levels by causing a greater decrease than a placebo at a specific timepoint after administration. Similarly, an “increase” in the level of a particular lipid or other biomarker may refer to either an increase versus baseline or an increase versus placebo.

A “therapeutically effective amount” of a composition as used herein is an amount of a composition that produces a desired therapeutic effect in a subject, such as treating a target condition. The precise therapeutically effective amount is an amount of the composition that will yield the most effective results in terms of therapeutic efficacy in a given subject. This amount will vary depending upon a variety of factors, including but not limited to the characteristics of the therapeutic composition (including, e.g., activity, pharmacokinetics, pharmacodynamics, and bioavailability), the physiological condition of the subject (including, e.g., age, body weight, sex, disease type and stage, medical history, general physical condition, responsiveness to a given dosage, and other present medications), the nature of the pharmaceutically acceptable carrier or carriers in the composition, and the route of administration. One skilled in the clinical and pharmacological arts will be able to determine a therapeutically effective amount through routine experimentation, namely by monitoring a subject's response to administration of a composition and adjusting the dosage accordingly. For additional guidance, see, e.g., Remington: The Science and Practice of Pharmacy, 21^{st} Edition, Univ. of Sciences in Philadelphia (USIP), Lippincott Williams & Wilkins, Philadelphia, Pa., 2005, and Goodman & Gilman's The Pharmacological Basis of Therapeutics, 11th Edition, McGraw-Hill, New York, N.Y., 2006, the entire disclosures of which are incorporated by reference herein.

A “pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. Such a carrier may comprise, for example, a liquid, gel, solid, or semi-solid filler, solvent, surfactant, diluent, excipient, adjuvant, binder, buffer, dissolution aid, solvent, encapsulating material, sequestering agent, dispersing agent, preservative, lubricant, disintegrant, thickener, emulsifier, antimicrobial agent, antioxidant, stabilizing agent, coloring agent, flavoring agent, or some combination thereof. Each component of the carrier must be “pharmaceutically acceptable” in that it must be compatible with the other ingredients of the composition and must be suitable for contact with any tissue, organ, or portion of the body that it may encounter, meaning that it must not carry a risk of toxicity, irritation, allergic response, immunogenicity, or any other complication that excessively outweighs its therapeutic benefits. Examples of pharmaceutically acceptable carriers for use in the presently disclosed pharmaceutical compositions include, but are not limited to, diluents such as microcrystalline cellulose or lactose (e.g., anhydrous lactose, lactose fast flo), binders such as gelatin, polyethylene glycol, wax, microcrystalline cellulose, synthetic gums such as polyvinylpyrrolidone, or cellulosic polymers such as hydroxypropyl cellulose (e.g., hydroxypropyl methylcellulose (HPMC)), lubricants such as magnesium stearate, calcium stearate, stearic acid, or microcrystalline cellulose, disintegrants such as starches, cross-linked polymers, or celluloses (e.g., croscarmellose sodium (CCNa), fillers such as silicon dioxide, titanium dioxide, microcrystalline cellulose, or powdered cellulose, surfactants or emulsifiers such as polysorbates (e.g., Polysorbate 20, 40, 60, or 80; Span 20, 40, 60, 65, or 80), antioxidant agents such as butylated hydroxyanisole (BHA), butylated hydroxytoluene (BHT), propyl gallate, or ascorbic acid (either free acid or salt forms thereof), buffers such as phosphate or citrate buffers, sequestering agents such as ethylenediaminetetraacetic acid (EDTA), ethylene glycol tetraacetic acid (EGTA), or edetate disodium, dispersing agents such as sodium carboxymethylcelluose, hydroxypropyl methylcellulose, povidone, or polyvinylpyrrolidone, dissolution aids such as calcium carbonate, and excipients such as water, saline, dextrose, glycerol, or ethanol, citric acid, calcium metabisulfite, lactic acid, malic acid, succinic acid, or tartaric acid.

Elevated levels of “bad” lipoproteins such as LDL, VLDL, and TG and low levels of “good” lipoproteins such as HDL are among the hallmarks of CVD. Reduction of TG levels and increasing of HDL levels have been shown to delay the onset and decrease the progression of atherosclerosis, and to decrease both the likelihood of developing CVD and the severity of CVD. A variety of therapeutic options for treating CVD have therefore focused on lowering levels of one or more bad lipoproteins or increasing levels of HDL.

A variety of compounds have been identified for treating CVD by decreasing bad lipoprotein levels and/or increasing good lipoprotein levels. These compounds include statins, niacin drugs, bile acid sequestrants such as cholestyramine resin (Questran®, Prevalite®), colestipol hydrochloride (Colestid®), or colesevelam hydrochloride (WelChol®, Cholestagel®), fibrates such as bezafibrate (Bezalip®), ciprofibrate (Modalim®), clofibrate, gemfibrozil (Lopid®), or fenofibrate (Antara®, TriCor®, ABT-335), cholesterol absorption inhibitors such as ezetimibe (Zetia®), AVE 5530, or MD-0727, cholesteryl ester transfer protein (CETP) inhibitors such as JTT-705/RO4607381 (R1658), CP-529414 (Torcetrapib®), or MK-0859, microsomal triglyceride transfer protein (MTP) inhibitors such as AEGR-733 and AEGR-733 combinations (e.g., AEGR-733 plus ezetimibe), squalene synthase inhibitors such as lapaquistat acetate (TAK-475) and lapaquistat acetate combinations (e.g., TAK-475 plus one or more statins), and other miscellaneous compounds such as dextrothyroxine, ISIS 301012, cardioprotectants such as MC-1 antibody, glycoprotein IIb/IIIa inhibitors such as tirofiban hydrochloride (Aggrastat®), TG100-115, AEGR 773, AEGR 427, stanols, and sterols.

Statins are among the most popular and effective therapeutic options for lowering LDL and TG levels. Statins are a class of compounds that inhibit HMG-CoA reductase from catalyzing the conversion of HMG-CoA to mevalonate, a rate-limiting step in the cholesterol biosynthetic pathway. In so doing, statins inhibit cholesterol biosynthesis and prevent the build-up of arterial plaque. In addition to significantly decreasing blood LDL levels and moderately decreasing blood TG levels, it has been proposed that statin administration may prevent CVD by improving endothelial function, modulating inflammatory responses, maintaining plaque stability, and preventing thrombus formation.

Niacin drugs are currently the most effective therapeutic known for raising HDL levels (Richman 2007). Niacin (also known as nicotinic acid, vitamin B₃, or pyridine-3-carboxylic acid) has also been shown to lower TG, VLDL, and LDL levels (Pike 2005; Offermanns 2006). Niacin has the structure:

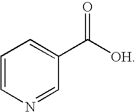

In subjects with stable CAD who were simultaneously being treated with statins, niacin administration has been shown to increase HDL levels and decrease TG, Lp-PLA₂, and CRP levels (Kuvin 2006). With regard to lipoprotein particle size, niacin administration increases large particle HDL levels while decreasing small particle HDL levels (Kuvin 2006).

On the basis of epidemiologic data, it has been predicted that every 1% decrease in LDL level results in a 1.0 to 1.5% decrease in the risk of major cardiovascular events (Assmann 1998). Similarly, each increase of 1 mg per deciliter in HDL level has been predicted to result in a two to four percent reduction in the risk of cardiac events (Gordon 1989). Since the effects of LDL lowering and HDL boosting are independent of one another, co-administration of an agent that lowers LDL with an agent that increases HDL is generally expected to produce an additive decrease in cardiovascular event risk. This is the basis for various combination therapies that utilize niacin plus one or more compounds that lower LDL, such as for example Simcor® (niacin plus simvastatin) and Advicor® (niacin plus lovastatin).

Previous studies have established that administration of sPLA₂ inhibitors results in a decrease in total cholesterol, LDL, total LDL particle, and small LDL particle levels in subjects with CAD (WO2008/137803). In addition, these studies established that co-administration of an sPLA₂ inhibitor with one or more statins decreases LDL and small LDL particle levels in a synergistic manner (i.e., the combined effect of A-002 and statin on LDL and small LDL particle levels is greater than the expected additive effect of administering A-002 and statin separately). This effect was not limited to a particular statin, but instead was observed across the statin subpopulation as a whole. Although these studies found a synergistic interaction between sPLA₂ inhibitors and statins on lipid levels, they did not evaluate the potential synergy between sPLA₂ inhibitors and other lipid-lowering agents.

As disclosed herein, studies were performed to determine whether sPLA₂ inhibitors are capable of acting in a synergistic manner with niacin drugs. Lipid levels were measured in subjects receiving sPLA₂ inhibitors, one or more niacin drugs, or sPLA₂ inhibitors in combination with one or more niacin drugs. Co-administration of sPLA₂ inhibitor and niacin drugs caused an unexpected synergistic decrease in TG levels and an unexpected synergistic increase in HDL levels. Therefore, provided herein in certain embodiments are compositions, methods, and kits for treating CVD, decreasing TG levels, increasing HDL levels, and increasing HDL/LDL ratio using a combination of one or more sPLA₂ inhibitors and one or more niacin drugs.

In certain embodiments, an sPLA₂ inhibitor for use in the methods, compositions, and kits disclosed herein may be an indole-based sPLA₂ inhibitor, meaning that the compound contains an indole nucleus having the structure:

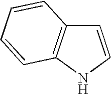

A variety of indole-based sPLA₂ inhibitors are known in the art. For example, indole-based sPLA₂ inhibitors that may be used in conjunction with the present invention include but are not limited to those set forth in U.S. Pat. Nos. 5,654,326 (Bach); 5,733,923 (Bach); 5,919,810 (Bach); 5,919,943 (Bach); 6,175,021 (Bach); 6,177,440 (Bach); 6,274,578 (Denney); and 6,433,001 (Bach), the entire disclosures of which are incorporated by reference herein. Methods of making indole-based sPLA₂ inhibitors are set forth in, for example, U.S. Pat. Nos. 5,986,106 (Khau); 6,265,591 (Anderson); and 6,380,397 (Anderson), the entire disclosures of which are incorporated by reference herein. sPLA₂ inhibitors for use in the present invention may be generated using these synthesis methods, or using any other synthesis method known in the art. In certain embodiments, sPLA₂ inhibitors for use in the present invention may be sPLA₂ type IIA, type V, and/or type X inhibitors. Various examples of indole-based sPLA₂ inhibitors are set forth below. These examples are merely provided as illustrations of the types of inhibitors that may be used in conjunction with the methods and compositions disclosed herein, and as such are not meant to be limiting. One of ordinary skill in the art will recognize that a variety of other indole-based sPLA₂ inhibitors may be used.

In certain embodiments, sPLA₂ inhibitors for use in the current invention are 1H-indole-3-glyoxylamide compounds having the structure:

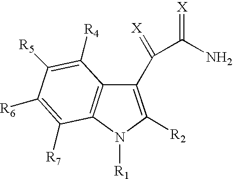

wherein:
each X is independently oxygen or sulfur;
R₁ is selected from the group consisting of (a), (b), and (c), wherein:

(a) is C₇-C₂₀ alkyl, C₇-C₂₀ alkenyl, C₇-C₂₀ alkynyl, carbocyclic radicals, or heterocyclic radicals;

(b) is a member of (a) substituted with one or more independently selected non-interfering substituents; and

(c) is the group -(L)-R₈₀, where, -(L)- is a divalent linking group of 1 to 12 atoms selected from carbon, hydrogen, oxygen, nitrogen, and sulfur, wherein the combination of atoms in -(L)- are selected from the group consisting of (i) carbon and hydrogen only, (ii) sulfur only, (iii) oxygen only, (iv) nitrogen and hydrogen only, (v) carbon, hydrogen, and sulfur only, and (vi) carbon, hydrogen, and oxygen only; and where R₈₀ is a group selected from (a) or (b);

R₂ is hydrogen, halo, C₁-C₃ alkyl, C₃-C₄ cycloalkyl, C₃-C₄ cycloalkenyl, —O—(C₁-C₂ alkyl), —S—(C₁-C₂ alkyl), or a non-interfering substituent having a total of 1 to 3 atoms other than hydrogen;
R₄ and R₅ are independently selected from the group consisting of hydrogen, a non-interfering substituent, and -(Lₐ)-(acidic group), wherein -(Lₐ)- is an acid linker having an acid linker length of 1 to 4; provided that at least one of R₄ and R₅ must be -(Lₐ)-(acidic group);
R₆ and R₇ are each independently selected from hydrogen, non-interfering substituents, carbocyclic radicals, carbocyclic radicals substituted with non-interfering substituents, heterocyclic radicals, and heterocyclic radicals substituted with non-interfering substituents;
provided that for any of the groups R₁, R₆, and R₇, the carbocyclic radical is selected from the group consisting of cycloalkyl, cycloalkenyl, phenyl, naphthyl, norbornanyl, bicycloheptadienyl, toluoyl, xylenyl, indenyl, stilbenzyl, terphenylyl, diphenylethylenyl, phenyl-cyclohexenly, acenaphthylenyl, and anthracenyl, biphenyl, bibenzylyl and related bibenzylyl homologues represented by the formula (bb),

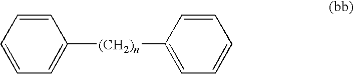

where n is a number from 1 to 8; provided, that for any of the groups R₁, R₆, and R₇, the heterocyclic radical is selected from the group consisting of pyrrolyl, furanyl, thiophenyl, pyrazolyl, imidazolyl, phenylimidazolyl, triazolyl, isoxazolyl, oxazolyl, thiazolyl, thiadiazolyl, indolyl, carbazolyl, norharmanyl, azaindolyl, benzofuranyl, dibenzofuranyl, thianaphtheneyl, dibenzothiophenyl, indazolyl, imidazo(1.2-A)pyridinyl, benzotriazolyl, anthranilyl, 1,2-benzisoxazolyl, benzoxazolyl, benzotriazolyl, purinyl, pryidinyl, dipyridylyl. phenylpyridinyl, benzylpyridinyl, pyrimidinyl, phenylpyrimidinyl, pyrazinyl, 1,3,5-triazinyl, quinolinyl, phthalazinyl, quinazolinyl, and quinoxalinyl; and provided that for the groups R₁, R₂, R₄, R₅, R₆, and R₇ the non-interfering substituent is selected from the group consisting of C₁-C₆ alkyl, C₂-C₆ alkenyl, C₂-C₆ alkynyl, C₇-C₁₂ aralkyl, C₇-C₁₂ alkaryl, C₃-C₈ cycloalkyl, C₃-C₈ cycloalkenyl, phenyl, toluoyl, xylenyl, biphenyl, C₁-C₆ alkoxy, C₂-C₆ alkenyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₂-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, —C(O)O(C₁-C₆ alkyl), —(CH₂)ₙ—O—(C₁-C₆ alkyl), benzyloxy, phenoxy, phenylthio, —(CONHSO₂R), —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl, where n is from 1 to 8;
and pharmaceutically acceptable salts, solvates, prodrug derivatives, racemates, tautomers, or optical isomers thereof.

In certain of these embodiments, -(L)- has the formula:

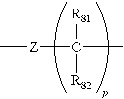

wherein R₈₁ and R₈₂ are each independently selected from the group consisting of hydrogen, C₁-C₁₀ alkyl, carboxy, carbalkoxy, and halo; p is a number from 1 to 5; and Z is selected from the group consisting of a bond, —(CH₂)—, —O—, —N(C₁-C₁₀ alkyl)-, —NH—, and —S—.

In certain of these embodiments wherein R₄ is -(Lₐ)-(acidic group), the acid linker -(Lₐ)- has the formula:

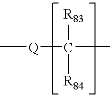

wherein Q is selected from the group consisting of —(CH₂)—, —O—, —NH—, and —S—; and R₈₃ and R₈₄ are each independently selected from the group consisting of hydrogen, C₁-C₁₀ alkyl, aryl, C₁-C₁₀ alkaryl, C₁-C₁₀ aralkyl, hydroxy, and halo.

In certain of these embodiments wherein R₅ is -(Lₐ)-(acidic group), the acid linker -(Lₐ)- has the formula:

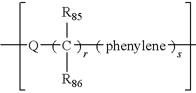

wherein r is a number from 2 to 7; s is 0 or 1; Q is selected from the group consisting of —(CH₂)—, —O—, —NH—, and —S—; and R₈₅ and R₈₆ are each independently selected from the group consisting of hydrogen, C₁-C₁₀ alkyl, aryl, C₁-C₁₀ alkaryl, C₁-C₁₀ aralkyl, carboxy, carbalkoxy, and halo.

In certain embodiments, a 1H-indole-3-glyoxylamide compound for use in the present invention is selected from the group consisting of: ((3-(2-Amino-1,2-dioxoethyl)-2-ethyl-1-(phenylmethyl)-1H-indol-4-yl)oxy)acetic acid; [[3-(2-Amino-1,2-dioxoethyl)-2-ethyl-1-(phenylmethyl)-1H-indol-4-yl]oxy]acetic acid methyl ester; ((3-(2-Amino-1,2-dioxoethyl)-2-methyl-1-(phenylmethyl)-1H-indol-4-yl)oxy)acetic acid; dl-2-((3-(2-Amino-1,2-dioxoethyl)-2-methyl-1-(phenylmethyl)-1H-indol-4-yl) oxy)propanoic acid; ((3-(2-Amino-1,2-dioxoethyl)-1-((1,1′-biphenyl)-2-ylmethyl)-2-methyl-1H-indol-4-yl)oxy)acetic acid; ((3-(2-Amino-1,2-dioxoethyl)-1-((1,1′-biphenyl)-3-ylmethyl)-2-methyl-1H-indol-4-yl)oxy)acetic acid; ((3-(2-Amino-1,2-dioxoethyl)-1-((1,1′-biphenyl)-4-ylmethyl)-2-methyl-1H-indol-4-yl)oxy)acetic acid; ((3-(2-Amino-1,2-dioxoethyl)-1-((2,6-dichlorophenyl)methyl)-2-methyl-1H-indol-4-yl)oxy)acetic acid; ((3-(2-Amino-1,2-dioxoethyl)-1-(4(-fluorophenyl)methyl)-2-methyl-1H-indol-4-yl)oxy)acetic acid; ((3-(2-Amino-1,2-dioxoethyl)-2-methyl-1-((1-naphthalenyl)methyl)-1H-indol-4-yl)oxy)acetic acid; ((3-(2-Amino-1,2-dioxoethyl)-1-(3-chlorophenyl)methyl)-2-ethyl-1H-indol-4-yl)oxy)acetic acid; ((3-(2-Amino-1,2-dioxoethyl)-1-((1,1′-biphenyl)-2-ylmethyl)-2-ethyl-1H-indol-4-yl)oxy)acetic acid; ((3-(2-amino-1,2-dioxoethyl)-1-((1,1′-biphenyl)-2-ylmethyl)-2-propyl-1H-indol-4-yl)oxy)acetic acid; ((3-(2-Amino-1,2-dioxoethyl)-2-cyclopropyl-1-(phenylmethyl)-1H-indol-4-yl) oxy)acetic acid; ((3-(2-Amino-1,2-dioxoethyl)-1-((1,1′-biphenyl)-2-ylmethyl)-2-cyclopropyl-1H-indol-4-yl)oxy)acetic acid; and 4-((3-(2-Amino-1,2-dioxoethyl)-2-ethyl-1-(phenylmethyl)-1H-indol-5-yl)oxy) butanoic acid, or pharmaceutically acceptable salts, solvates, prodrug derivatives, racemates, tautomers, or optical isomers thereof.

In certain embodiments, sPLA₂ inhibitors for use in the current invention are 1H-indole-3-glyoxylamide compounds having the structure:

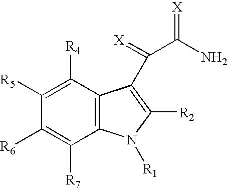

wherein:
both X are oxygen;
R₁ is selected from the group consisting of:

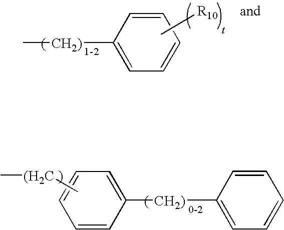

wherein R₁₀ is a radical independently selected from halo, C₁-C₁₀ alkoxy, —S—(C₁-C₁₀ alkyl), and C₁-C₁₀ haloalkyl, and t is a number from 0 to 5;
R₂ is selected from the group consisting of halo, cyclopropyl, methyl, ethyl, and propyl;
R₄ and R₅ are independently, selected from the group consisting of hydrogen, a non-interfering substituent, and -(Lₐ)-(acidic group), wherein -(Lₐ)- is an acid linker; provided that the acid linker -(Lₐ)- for R₄ is selected from the group consisting of:

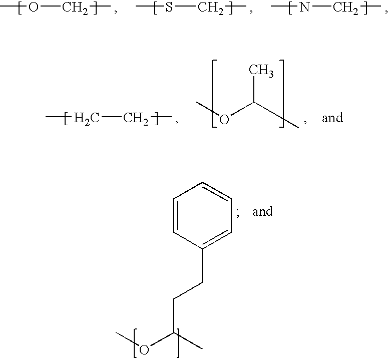

provided that the acid linker -(Lₐ)- for R₅ is selected from the group consisting of:

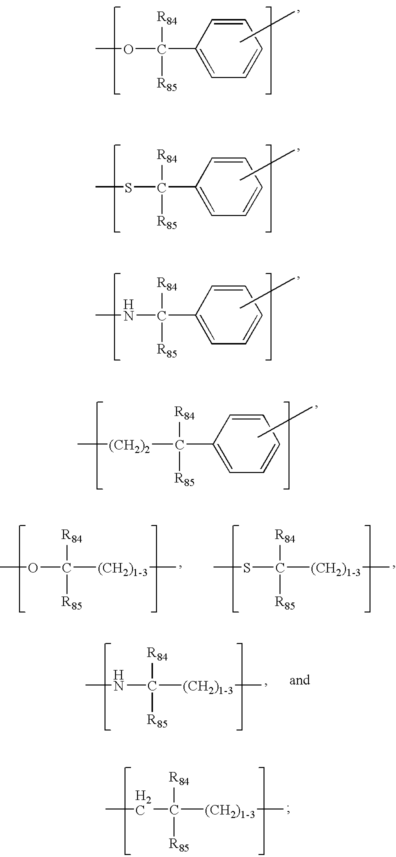

wherein R₈₄ and R₈₅ are each independently selected from the group consisting of hydrogen, C₁-C₁₀ alkyl, aryl, C₁-C₁₀ alkaryl, C₁-C₁₀ aralkyl, carboxy, carbalkoxy, and halo; provided that at least one of R₄ and R₅ must be -(Lₐ)-(acidic group), and (acidic group) on -(Lₐ)-(acidic group) of R₄ or R₅ is selected from —CO₂H, —SO₃H, or —P(O)(OH)₂;
R₆ and R₇ are each independently selected from the group consisting of hydrogen and non-interfering substituents, with the non-interfering substituents being selected from the group consisting of: C₁-C₆ alkyl, C₂-C₆ alkenyl, C₂-C₆ alkynyl, C₇-C₁₂ aralkyl, C₇-C₁₂ alkaryl, C₃-C₈ cycloalkyl, C₃-C₈ cycloalkenyl, phenyl, toluoyl, xylenyl, biphenyl, C₁-C₆ alkoxy, C₂-C₆ alkenyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₂-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, —C(O)O(C₁-C₆ alkyl), —(CH₂)ₙ—O—(C₁-C₆ alkyl), benzyloxy, phenoxy, phenylthio, —(CONHSO₂R), —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl; wherein n is from 1 to 8;
and pharmaceutically acceptable salts, solvates, prodrug derivatives, racemates, tautomers, or optical isomers thereof.

In certain embodiments, 1H-indole-3-glyoxylamide compounds for use in the present invention are selected from the group consisting of: ((3-(2-Amino-1,2-dioxoethyl)-2-methyl-1-(phenylmethyl)-1H-indol-4-yl)oxy)acetic acid; ((3-(2-Amino-1,2-dioxoethyl)-2-methyl-1-(phenylmethyl)-1H-indol-4-yl)oxy)acetic acid methyl ester; dl-2-((3-(2-Amino-1,2-dioxoethyl)-2-methyl-1-(phenylmethyl)-1H-indol-4-yl) oxy)propanoic acid; dl-2-((3-(2-Amino-1,2-dioxoethyl)-2-methyl-1-(phenylmethyl)-1H-indol-4-yl) oxy)propanoic acid methyl ester; ((3-(2-Amino-1,2-dioxoethyl)-1-((1,1′-biphenyl)-2-ylmethyl)-2-methyl-1H-indol-4-yl)oxy)acetic acid; ((3-(2-Amino-1,2-dioxoethyl)-1-((1,1′-biphenyl)-2-ylmethyl)-2-methyl-1H-indol-4-yl)oxy)acetic acid methyl ester; ((3-(2-Amino-1,2-dioxoethyl)-1*(1,1′-biphenyl)-3-ylmethyl)-2-methyl-1H-indol-4-yl)oxy)acetic acid; ((3-(2-Amino-1,2-dioxoethyl)-1-((1,1′-biphenyl)-3-ylmethyl)-2-methyl-1H-indol-4-yl)oxy)acetic acid methyl ester; ((3-(2-Amino-1,2-dioxoethyl)-1-((1,1′-biphenyl)-4-ylmethyl)-2-methyl-1H-indol-4-yl)oxy)acetic acid; ((3-(2-Amino-1,2-dioxoethyl)-1-((1,1′-biphenyl)-4-ylmethyl)-2-methyl-1H-indol-4-yl)oxy)acetic acid methyl ester; ((3-(2-Amino-1,2-dioxoethyl)-1-((2,6-dichlorophenyl)methyl)-2-methyl-1H-indol-4-yl)oxy)acetic acid; ((3-(2-Amino-1,2-dioxoethyl)-1-((2,6-dichlorophenyl)methyl)-2-methyl-1H-indol-4-yl)oxy)acetic acid methyl ester; ((3-(2-Amino-1,2-dioxoethyl)-1-(4(-fluorophenyl)methyl)-2-methyl-1H-indol-4-yl)oxy)acetic acid; ((3-(2-Amino-1,2-dioxoethyl)-1-(4(-fluorophenyl)methyl)-2-methyl-1H-indol-4-yl)oxy)acetic acid methyl ester; ((3-(2-Amino-1,2-dioxoethyl)-2-methyl-1-((1-naphthalenyl)methyl)-1H-indol-4-yl)oxy)acetic acid; ((3-(2-Amino-1,2-dioxoethyl)-2-methyl-1-((1-naphthalenyl)methyl)-1H-indol-4-yl)oxy)acetic acid methyl ester; ((3-(2-Amino-1,2-dioxoethyl)-1-(3-chlorophenyl)methyl)-2-ethyl-1H-indol-4-yl)oxy)acetic acid; ((3-(2-Amino-1,2-dioxoethyl)-1-((3-chlorophenyl)methyl)-2-ethyl-1H-indol-4-yl)oxy)acetic acid methyl ester; ((3-(2-Amino-1,2-dioxoethyl)-1-((1,1′-biphenyl)-2-ylmethyl)-2-ethyl-1H-indol-4-yl)oxy)acetic acid; ((3-(2-Amino-1,2-dioxoethyl)-1-((1,1′-biphenyl)-2-ylmethyl)-2-ethyl-1H-indol-4-yl)oxy)acetic acid methyl ester; ((3-(2-amino-1,2-dioxoethyl)-1-((1,1′-biphenyl)-2-ylmethyl)-2-propyl-1H-indol-4-yl)oxy)acetic acid; ((3-(2-amino-1,2-dioxoethyl)-1-((1,1′-biphenyl)-2-ylmethyl)-2-propyl-1H-indol-4-yl)oxy)acetic acid methyl ester; ((3-(2-Amino-1,2-dioxoethyl)-2-cyclopropyl-1-(phenylmethyl)-1H-indol-4-yl) oxy)acetic acid; ((3-(2-Amino-1,2-dioxoethyl)-2-cyclopropyl-1-(phenylmethyl)-1H-indol-4-yl) oxy)acetic acid methyl ester; ((3-(2-Amino-1,2-dioxoethyl)-1-(1,1′-biphenyl)-2-ylmethyl)-2-cyclopropyl-1H-indol-4-yl)oxy)acetic acid; ((3-(2-Amino-1,2-dioxoethyl)-1-((1,1′-biphenyl)-2-ylmethyl)-2-cyclopropyl-1H-indol-4-yl)oxy)acetic acid methyl ester; 4-((3-(2-Amino-1,2-dioxoethyl)-2-ethyl-1-(phenylmethyl)-1H-indol-5-yl)oxy) butanoic acid; 4-((3-(2-Amino-1,2-dioxoethyl)-2-ethyl-1-(phenylmethyl)-1H-indol-5-yl)oxy) butanoic acid tert-butyl ester, or pharmaceutically acceptable salts, solvates, prodrug derivatives, racemates, tautomers, or optical isomers thereof.

In certain embodiments, sPLA₂ inhibitors for use in the current invention are 1H-indole-3-glyoxylamide compounds having the structure:

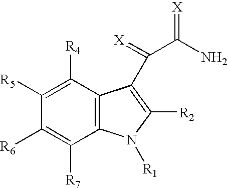

wherein:

each X is independently oxygen or sulfur;

R₁ is selected from groups (a), (b), and (c) wherein:

(a) is C₇-C₂₀ alkyl, C₇-C₂₀ alkenyl, C₇-C₂₀ alkynyl, carbocyclic radical, or heterocyclic radical;

(b) is a member of (a) substituted with one or more independently selected non-interfering substituents; and

(c) is the group -(L)-R₈₀, wherein -(L)- is a divalent linking group of 1 to 12 atoms selected from carbon, hydrogen, oxygen, nitrogen, and sulfur; wherein the combination of atoms in -(L)- are selected from the group consisting of (i) carbon and hydrogen only, (ii) sulfur only, (iii) oxygen only, (iv) nitrogen and hydrogen only, (v) carbon, hydrogen, and sulfur only, and (vi) and carbon, hydrogen, and oxygen only; and where R₈₀ is a group selected from (a) or (b);

R₂ is selected from the group consisting of hydrogen, halo, C₁-C₃ alkyl, C₃-C₄ cycloalkyl, C₃-C₄ cycloalkenyl, —O—(C₁-C₂ alkyl), —S—(C₁-C₂ alkyl), and a non-interfering substituent having a total of 1 to 3 atoms other than hydrogen;
R₄ and R₅ are independently selected from the group consisting of hydrogen, a non-interfering substituent, and the group -(Lₐ)-(acidic group), wherein -(Lₐ)- is an acid linker having an acid linker length of 1 to 4; provided that at least one of R₄ and R₅ is -(Lₐ)-(acidic group);
R₆ and R₇ are each independently selected from the group consisting of hydrogen, non-interfering substituents, carbocyclic radicals, carbocyclic radicals substituted with non-interfering substituents, heterocyclic radicals, and heterocyclic radicals substituted with non-interfering substituents;
and pharmaceutically acceptable salts, solvates, prodrug derivatives, racemates, tautomers, or optical isomers thereof.

In certain embodiments, sPLA₂ inhibitors for use in the current invention are methyl ester prodrug derivatives of 1H-indole-3-glyoxylamide compounds having the structure:

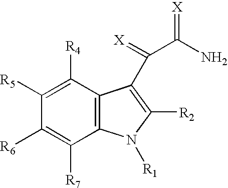

wherein:
both X are oxygen;
R₁ is selected from the group consisting of:

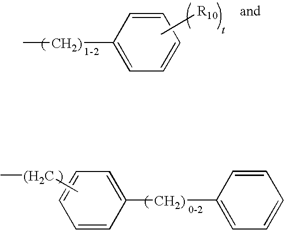

wherein R₁₀ is a radical independently selected from halo, C₁-C₁₀ alkyl, C₁-C₁₀ alkoxy, —S—(C₁-C₁₀ alkyl), and C₁-C₁₀ haloalkyl, and t is a number from 0 to 5;
R₂ is selected from the group consisting of halo, cyclopropyl, methyl, ethyl, and propyl;
R₄ and R₅ are independently selected from the group consisting of hydrogen, a non-interfering substituent, and -(Lₐ)-(acidic group), wherein -(Lₐ)- is an acid linker; provided that the acid linker -(Lₐ)- for R₄ is selected from the group consisting of:

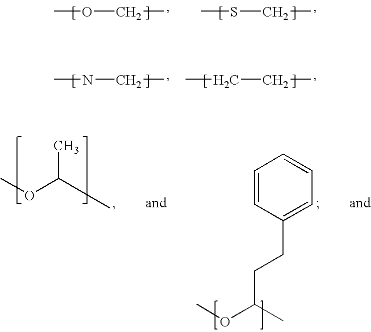

provided that the acid linker -(Lₐ)- for R₅ is selected from the group consisting of:

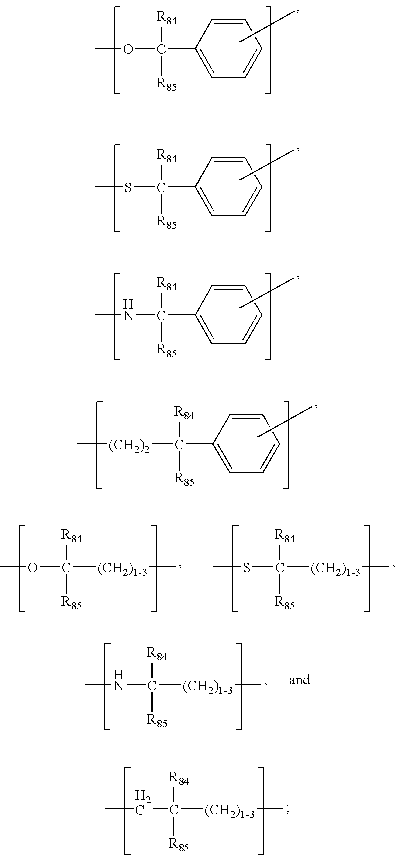

wherein R₈₄ and R₈₅ are each independently selected from the group consisting of hydrogen, C₁-C₁₀ alkyl, aryl, C₁-C₁₀ alkaryl, C₁-C₁₀ aralkyl, carboxy, carbalkoxy, and halo; provided that at least one of R₄ and R₅ must be -(Lₐ)-(acidic group), and (acidic group) on -(Lₐ)-(acidic group) of R₄ or R₅ is selected from —CO₂H, —SO₃H, or —P(O)(OH)₂;
R₆ and R₇ are each independently selected from the group consisting of hydrogen and non-interfering substituents, with the non-interfering substituents being selected from the group consisting of: C₁-C₆ alkyl, C₂-C₆ alkenyl, C₂-C₆ alkynyl, C₇-C₁₂ aralkyl, C₇-C₁₂ alkaryl, C₃-C₈ cycloalkyl, C₃-C₈ cycloalkenyl, phenyl, toluoyl, xylenyl, biphenyl, C₁-C₆ alkoxy, C₂-C₆ alkenyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₂-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, —C(O)O(C₁-C₆ alkyl), —(CH₂)ₙ—O—(C₁-C₆ alkyl), benzyloxy, phenoxy, phenylthio, —(CONHSO₂R), —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl; wherein n is from 1 to 8;
and pharmaceutically acceptable salts, solvates, prodrug derivatives, racemates, tautomers, or optical isomers thereof.

In certain embodiments, sPLA₂ inhibitors for use in the current invention are (acyloxy) alkyl ester prodrug derivatives of 1H-indole-3-glyoxylamide compounds having the structure:

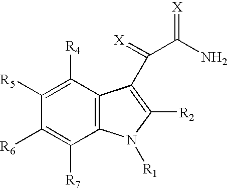

wherein:
both X are oxygen;
R₁ is selected from the group consisting of:

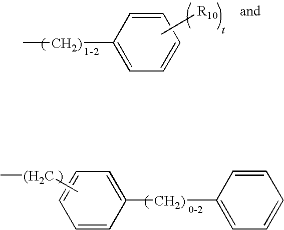

wherein R₁₀ is a radical independently selected from halo, C₁-C₁₀ alkyl, C₁-C₁₀ alkoxy, —S—(C₁-C₁₀ alkyl), and C₁-C₁₀ haloalkyl, and t is a number from 0 to 5;
R₂ is selected from the group consisting of halo, cyclopropyl, methyl, ethyl, and propyl;
R₄ and R₅ are independently selected from the group consisting of hydrogen, a non-interfering substituent, and -(Lₐ)-(acidic group), wherein -(Lₐ)- is an acid linker; provided that the acid linker -(Lₐ)- for R₄ is selected from the group consisting of:

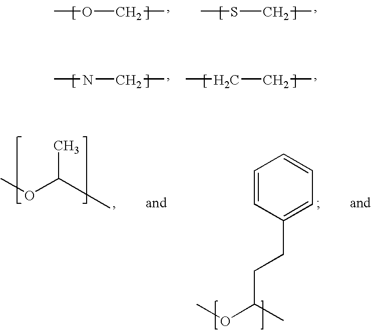

provided that the acid linker -(Lₐ)- for R₅ is selected from the group consisting of:

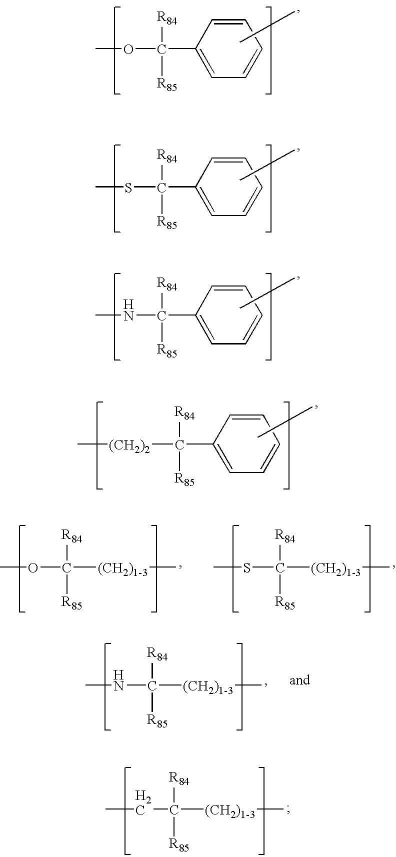

wherein R₈₄ and R₈₅ are each independently selected from the group consisting of hydrogen, C₁-C₁₀ alkyl, aryl, C₁-C₁₀ alkaryl, C₁-C₁₀ aralkyl, carboxy, carbalkoxy, and halo; provided that at least one of R₄ and R₅ must be -(Lₐ)-(acidic group), and (acidic group) on -(Lₐ)-(acidic group) of R₄ or R₅ is selected from —CO₂H, —SO₃H, or —P(O)(OH)₂;
R₆ and R₇ are each independently selected from the group consisting of hydrogen and non-interfering substituents, with the non-interfering substituents being selected from the group consisting of: C₁-C₆ alkyl, C₂-C₆ alkenyl, C₂-C₆ alkynyl, C₇-C₁₂ aralkyl, C₇-C₁₂ alkaryl, C₃-C₈ cycloalkyl, C₃-C₈ cycloalkenyl, phenyl, toluoyl, xylenyl, biphenyl, C₁-C₆ alkoxy, C₂-C₆ alkenyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₂-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, —C(O)O(C₁-C₆ alkyl), —(CH₂)ₙ—O—(C₁-C₆ alkyl), benzyloxy, phenoxy, phenylthio, —(CONHSO₂R), —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl; wherein n is from 1 to 8;
and pharmaceutically acceptable salts, solvates, prodrug derivatives, racemates, tautomers, or optical isomers thereof.

In certain embodiments, sPLA₂ inhibitors for use in the current invention are substituted tricyclics having the structure:

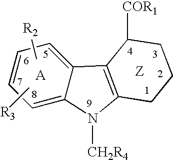

wherein:
R₁ is selected from the group consisting of —NHNH₂ and —NH₂;
R₂ is selected from the group consisting of —OH and —O(CH₂)ₘR₅; wherein R₅ is selected from the group consisting of H, —CO₂H, —CO₂(C₁-C₄ alkyl), —SO₃H, —SO₃(C₁-C₄ alkyl), tetrazolyl, —CN, —NH₂, —NHSO₂R₁₅, —CONHSO₂R₁₅, phenyl, phenyl substituted with —CO₂H or —CO₂(C₁-C₄)alkyl, and

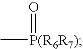

wherein R₆ and R₇ are each independently selected from the group consisting of —OH, —O(C₁-C₄)alkyl; R₁₅ is selected from the group consisting of —(C₁-C₆)alkyl and —CF₃; and m is 1-3;
R₃ is selected from the group consisting of H, —O(C₁-C₄)alkyl, halo, —(C₁-C₆)alkyl, phenyl, —(C₁-C₄)alkylphenyl, phenyl substituted with —(C₁-C₆)alkyl, halo, or —CF₃, —CH₂OSi(C₁-C₆)alkyl, furyl, thiophenyl, —(C₁-C₆)hydroxyalkyl, and —(CH₂)ₙR₈; wherein R₈ is selected from the group consisting of H, —CONH₂, —NR₉R₁₀, —CN, and phenyl;
wherein R₉ and R₁₀ are each independently —(C₁-C₄)alkyl or -phenyl(C₁-C₄)alkyl; and n is 1 to 8;
R₄ is selected from the group consisting of H, —(C₅-C₁₄)alkyl, —(C₃-C₁₄)cycloalkyl, pyridyl, phenyl, and phenyl substituted with —(C₁-C₆)alkyl, halo, —CF₃, —OCF₃, —(C₁-C₄)alkoxy, —CN, —(C₁-C₄)alkylthio, phenyl(C₁-C₄)alkyl, —(C₁-C₄)alkylphenyl, phenyl, phenoxy, or naphthyl;
A is selected from the group consisting of phenyl and pyridyl wherein the nitrogen is at the 5-, 6-, 7-, or 8-position;
Z is selected from the group consisting of cyclohexenyl, phenyl, pyridyl wherein the nitrogen is at the 1-, 2-, or 3-position, and a 6-membered heterocyclic ring having one heteroatom selected from the group consisting of sulfur and oxygen at the 1-, 2-, or 3-position and nitrogen at the 1-, 2-, 3-, or 4-position, or wherein one carbon on the heterocyclic ring is optionally substituted with ═O; and wherein one of A or Z is a heterocyclic ring;
and pharmaceutically acceptable salts, solvates, prodrug derivatives, racemates, tautomers, or optical isomers thereof.

In certain embodiments, sPLA₂ inhibitors for use in the current invention are substituted tricyclics having the structure:

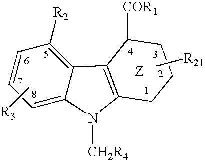

wherein:
Z is selected from the group consisting of cyclohexenyl and phenyl;
R₂₁ is a non-interfering substituent;

### R₁ is —NHNH₂ or —NH₂;

R₂ is selected from the group consisting of —OH and —O(CH₂)ₘR₅; wherein R₅ is selected from the group consisting of H, —CO₂H, —CONH₂, —CO₂(C₁-C₄ alkyl), —SO₃H, —SO₃(C₁-C₄ alkyl), tetrazolyl, —CN, —NH₂, —NHSO₂R₁₅, —CONHSO₂R₁₅, phenyl, phenyl substituted with —CO₂H or —CO₂(C₁-C₄)alkyl, and

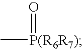

wherein R₆ and R₇ are each independently selected from the group consisting of —OH, —O(C₁-C₄)alkyl; R₁₅ is selected from the group consisting of —(C₁-C₆)alkyl and —CF₃; and m is 1-3;
R₃ selected from the group consisting of H, —O(C₁-C₄)alkyl, halo, —(C₁-C₆)alkyl, phenyl, —(C₁-C₄)alkylphenyl, phenyl substituted with —(C₁-C₆)alkyl, halo, or —CF₃, —CH₂OSi(C₁-C₆)alkyl, furyl, thiophenyl, —(C₁-C₆)hydroxyalkyl, and —(CH₂)ₙR₈; wherein R₈ is selected from the group consisting of H, —CONH₂, —NR₉R₁₀, —CN, and phenyl; R₉ and R₁₀ are each independently selected from the group consisting of H, —CF₃, phenyl, —(C₁-C₄)alkyl, —(C₁-C₄)alkylphenyl, and -phenyl(C₁-C₄)alkyl; and n is 1 to 8;
R₄ is selected from the group consisting of H, —(C₅-C₁₄)alkyl, —(C₃-C₁₄)cycloalkyl, pyridyl, phenyl, phenyl substituted with —(C₁-C₆)alkyl, halo, —CF₃, —OCF₃, —(C₁-C₄)alkoxy, —CN, —(C₁-C₄)alkylthio, -phenyl(C₁-C₄)alkyl, —(C₁-C₄)alkylphenyl, phenyl, phenoxy and naphthyl;
and pharmaceutically acceptable salts, solvates, prodrug derivatives, racemates, tautomers, or optical isomers thereof.

In certain embodiments, sPLA₂ inhibitors for use in the current invention are selected from the group consisting of: {9-[(phenyl)methyl]-5-carbamoylcarbazol-4-yl}oxyacetic acid; 9-benzyl-5,7-dimethoxy-1,2,3,4-tetrahydrocarbazole-4-carboxylic acid hydrazide; 9-benzyl-5,7-dimethoxy-1,2,3,4-tetrahydrocarbazole-4-carboxamide; [9-benzyl-4-carbamoyl-7-methoxy-1,2,3,4-tetrahydrocarbazol-5-yl]oxyacetic acid; [9-benzyl-4-carbamoyl-7-methoxycarbazol-5-yl]oxyacetic acid; methyl[9-benzyl-4-carbamoyl-7-methoxycarbazol-5-yl]oxyacetic acid; 9-benzyl-7-methoxy-5-cyanomethyloxy-1,2,3,4-tetrahydrocarbazole-4-carboxamide; 9-benzyl-7-methoxy-5-(1H-tetrazol-5-yl-methyl)oxy)-1,2,3,4-tetrahydrocarbazole-4-carboxamide; {9-[(phenyl)methyl]-5-carbamoyl-2-methyl-carbazol-4-yl}oxyacetic acid; {9-[(3-fluorophenyl)methyl]-5-carbamoyl-2-methylcarbazol-4-yl}oxyacetic acid; {9-[(3-methylphenyl)methyl]-5-carbamoyl-2-methylcarbazol-4-yl}oxyacetic acid; {9-[(phenyl)methyl]-5-carbamoyl-2-(4-trifluoromethylphenyl)-carbazol-4-yl}oxyacetic acid; 9-benzyl-5-(2-methanesulfonamido)ethyloxy-7-methoxy-1,2,3,4-tetrahydrocarbazole-4-carboxamide; 9-benzyl-4-(2-methanesulfonamido)ethyloxy-2-methoxycarbazole-5-carboxamide; 9-benzyl-4-(2-trifluoromethanesulfonamido)ethyloxy-2-methoxycarbazole-5-carboxamide; 9-benzyl-5-methanesulfonamidoylmethyloxy-7-methoxy-1,2,3,4-tetrahydrocarbazole-4-carboxamide; 9-benzyl-4-methanesulfonamidoylmethyloxy-carbazole-5-carboxamide; [5-carbamoyl-2-pentyl-9-(phenylmethyl)carbazol-4-yl]oxyacetic acid; [5-carbamoyl-2-(1-methylethyl)-9-(phenylmethyl)carbazol-4-yl]oxyacetic acid; [5-carbamoyl-9-(phenylmethyl)-2-[(tri(−1-methylethyl)silyl)oxymethyl]carbazol-4-yl]oxyacetic acid; [5-carbamoyl-2-phenyl-9-(phenylmethyl)carbazol-4-yl]oxyacetic acid; [5-carbamoyl-2-(4-chlorophenyl)-9-(phenylmethyl)carbazol-4-yl]oxyacetic acid; [5-carbamoyl-2-(2-furyl)-9-(phenylmethyl)carbazol-4-yl]oxyacetic acid; [5-carbamoyl-9-(phenylmethyl)-2-[(tri(−1-methylethyl)silyl)oxymethyl]carbazol-4-yl]oxyacetic acid; {9-[(2-Fluorophenyl)methyl]-5-carbamoylcarbazol-4-yl}oxyacetic acid; {9-[(2-trifluoromethylphenyl)methyl]-5-carbamoylcarbazol-4-yl}oxyacetic acid; {9-[(2-benzylphenyl)methyl]-5-carbamoylcarbazol-4-yl}oxyacetic acid; {9-[(1-naphthyl)methyl]-5-carbamoylcarbazol-4-yl}oxyacetic acid; {9-[(2-cyanophenyl)methyl]-5-carbamoylcarbazol-4-yl}oxyacetic acid; {9-[(3-cyanophenyl)methyl]-5-carbamoylcarbazol-4-yl}oxyacetic acid; {9-[(3,5-dimethylphenyl)methyl]-5-carbamoylcarbazol-4-yl}oxyacetic acid; {9-[(3-iodophenyl)methyl]-5-carbamoylcarbazol-4-yl}oxyacetic acid; {9-[(2-Chlorophenyl)methyl]-5-carbamoylcarbazol-4-yl}oxyacetic acid; {9-[(2,3-difluorophenyl)methyl]-5-carbamoylcarbazol-4-yl}oxyacetic acid; {9-[(2,6-difluorophenyl)methyl]-5-carbamoylcarbazol-4-yl}oxyacetic acid; {9-[(2,6-dichlorophenyl)methyl]-5-carbamoylcarbazol-4-yl}oxyacetic acid; {9-[(2-biphenyl)methyl]-5-carbamoylcarbazol-4-yl}oxyacetic acid; {9-[(2-Biphenyl)methyl]-5-carbamoylcarbazol-4-yl}oxyacetic acid methyl ester; [9-Benzyl-4-carbamoyl-1,2,3,4-tetrahydrocarbazol-5-yl]oxyacetic acid; {9-[(2-Pyridyl)methyl]-5-carbamoylcarbazol-4-yl}oxyacetic acid; {9-[(3-Pyridyl)methyl]-5-carbamoylcarbazol-4-yl}oxyacetic acid; [9-benzyl-4-carbamoyl-8-methyl-1,2,3,4-tetrahydrocarbazol-5-yl]oxyacetic acid; [9-benzyl-5-carbamoyl-1-methylcarbazol-4-yl]oxyacetic acid; [9-benzyl-4-carbamoyl-8-fluoro-1,2,3,4-tetrahydrocarbazol-5-yl]oxyacetic acid; [9-benzyl-4-carbamoyl-8-chloro-1,2,3,4-tetrahydrocarbazol-5-yl]oxyacetic acid; [5-carbamoyl-9-(phenylmethyl)-2-[[(propen-3-yl)oxy]methyl]carbazol-4-yl]oxyacetic acid; [5-carbamoyl-9-(phenylmethyl)-2-[(propyloxy)methyl]carbazol-4-yl]oxyacetic acid; 9-benzyl-7-methoxy-5-((carboxamidomethyl)oxy)-1,2,3,4-tetrahydrocarbazole-4-carboxamide; 9-benzyl-7-methoxy-5-cyanomethyloxy-carbazole-4-carboxamide; 9-benzyl-7-methoxy-5-((1H-tetrazol-5-yl-methyl)oxy)-carbazole-4-carboxamide; 9-benzyl-7-methoxy-5-((carboxamidomethyl)oxy)-carbazole-4-carboxamide; [9-Benzyl-4-carbamoyl-1,2,3,4-tetrahydrocarbazole-5-yl]oxyacetic acid; {9-[(phenyl)methyl]-5-carbamoyl-2-methyl-carbazol-4-yl}oxyacetic acid; {9-[(3-fluorophenyl)methyl]-5-carbamoyl-2-methylcarbazol-4-yl}oxyacetic acid; {9-[(3-methylphenyl)methyl]-5-carbamoyl-2-methylcarbazol-4-yl}oxyacetic acid; {9-[(phenyl)methyl]-5-carbamoyl-2-(4-trifluoromethylphenyl)-carbazol-4-yl}oxyacetic acid; 9-benzyl-5-(2-methanesulfonamido)ethyloxy-7-methoxy-1,2,3,4-tetrahydrocarbazole-4-carboxamide; 9-benzyl-4-(2-methanesulfonamido)ethyloxy-2-methoxycarbazole-5-carboxamide; 9-benzyl-4-(2-trifluoromethanesulfonamido)ethyloxy-2-methoxycarbazole-5-carboxamide; 9-benzyl-5-methanesulfonamidoylmethyloxy-7-methoxy-1,2,3,4-tetrahydrocarbazole-4-carboxamide; 9-benzyl-4-methanesulfonamidoylmethyloxy-carbazole-5-carboxamide; [5-carbamoyl-2-pentyl-9-(phenylmethyl)carbazol-4-yl]oxyacetic acid; [5-carbamoyl-2-(1-methylethyl)-9-(phenylmethyl)carbazol-4-yl]oxyacetic acid; [5-carbamoyl-9-(phenylmethyl)-2-[(tri(−1-methylethyl)silyl)oxymethyl]carbazol-4-yl]oxyacetic acid; [5-carbamoyl-2-phenyl-9-(phenylmethyl)carbazol-4-yl]oxyacetic acid; [5-carbamoyl-2-(4-chlorophenyl)-9-(phenylmethyl)carbazol-4-yl]oxyacetic acid; [5-carbamoyl-2-(2-furyl)-9-(phenylmethyl)carbazol-4-yl]oxyacetic acid; [5-carbamoyl-9-(phenylmethyl)-2-[(tri(−1-methylethyl)silyl)oxymethyl]carbazol-4-yl]oxyacetic acid; {9-[(3-fluorophenyl)methyl]-5-carbamoylcarbazol-4-yl}oxyacetic acid; {9-[(3-chlorophenyl)methyl]-5-carbamoylcarbazol-4-yl}oxyacetic acid; {9-[(3-phenoxyphenyl)methyl]-5-carbamoylcarbazol-4-yl}oxyacetic acid; {9-[(2-Fluorophenyl)methyl]-5-carbamoylcarbazol-4-yl}oxyacetic acid; {9-[(2-trifluoromethylphenyl)methyl]-5-carbamoylcarbazol-4-yl}oxyacetic acid; {9-[(2-benzylphenyl)methyl]-5-carbamoylcarbazol-4-yl}oxyacetic acid; {9-[(3-trifluoromethylphenyl)methyl]-5-carbamoylcarbazol-4-yl}oxyacetic acid; {9-[(1-naphthyl)methyl]-5-carbamoylcarbazol-4-yl}oxyacetic acid; {9-[(2-cyanophenyl)methyl]-5-carbamoylcarbazol-4-yl}oxyacetic acid; {9-[(3-cyanophenyl)methyl]-5-carbamoylcarbazol-4-yl}oxyacetic acid; {9-[(2-methylphenyl)methyl]-5-carbamoylcarbazol-4-yl}oxyacetic acid; {9-[(3-methylphenyl)methyl]-5-carbamoylcarbazol-4-yl}oxyacetic acid; {9-[(3,5-dimethylphenyl)methyl]-5-carbamoylcarbazol-4-yl}oxyacetic acid; {9-[(3-iodophenyl)methyl]-5-carbamoylcarbazol-4-yl}oxyacetic acid; {9-[(2-Chlorophenyl)methyl]-5-carbamoylcarbazol-4-yl}oxyacetic acid; {9-[(2,3-difluorophenyl)methyl]-5-carbamoylcarbazol-4-yl}oxyacetic acid; {9-[(2,6-difluorophenyl)methyl]-5-carbamoylcarbazol-4-yl}oxyacetic acid; {9-[(2,6-dichlorophenyl)methyl]-5-carbamoylcarbazol-4-yl}oxyacetic acid; {9-[(3-trifluoromethoxyphenyl)methyl]-5-carbamoylcarbazol-4-yl}oxyacetic acid; {9-[(2-biphenyl)methyl]-5-carbamoylcarbazol-4-yl}oxyacetic acid; {9-[(2-Biphenyl)methyl]-5-carbamoylcarbazol-4-yl}oxyacetic acid methyl ester; [9-Benzyl-4-carbamoyl-1,2,3,4-tetrahydrocarbazole-5-yl]oxyacetic acid; {9-[(2-Pyridyl)methyl]-5-carbamoylcarbazol-4-yl}oxyacetic acid; {9-[(3-Pyridyl)methyl]-5-carbamoylcarbazol-4-yl}oxyacetic acid; [9-benzyl-4-carbamoyl-8-methyl-1,2,3,4-tetrahydrocarbazol-5-yl]oxyacetic acid; [9-benzyl-5-carbamoyl-1-methylcarbazol-4-yl]oxyacetic acid; [9-benzyl-4-carbamoyl-8-fluoro-1,2,3,4-tetrahydrocarbazol-5-yl]oxyacetic acid; [9-benzyl-5-carbamoyl-1-fluorocarbazol-4-yl]oxyacetic acid; [9-benzyl-4-carbamoyl-8-chloro-1,2,3,4-tetrahydrocarbazol-5-yl]oxyacetic acid; [9-benzyl-5-carbamoyl-1-chlorocarbazol-4-yl]oxyacetic acid; [9-[(Cyclohexyl)methyl]-5-carbamoylcarbazol-4-yl]oxyacetic acid; [9-[-(Cyclopentyl)methyl]-5-carbamoylcarbazol-4-yl]oxyacetic acid; [5-carbamoyl-9-(phenylmethyl)-2-(2-thienyl)carbazol-4-yl]oxyacetic acid; [5-carbamoyl-9-(phenylmethyl)-2-[[(propen-3-yl)oxy]methyl]carbazol-4-yl]oxyacetic acid; [5-carbamoyl-9-(phenylmethyl)-2-[(propyloxy)methyl]carbazol-4-yl]oxyacetic acid; 9-benzyl-7-methoxy-5-((carboxamidomethyl)oxy)-1,2,3,4-tetrahydrocarbazole-4-carboxamide; 9-benzyl-7-methoxy-5-cyanomethyloxy-carbazole-4-carboxamide; 9-benzyl-7-methoxy-5-((1H-tetrazol-5-yl-methyl)oxy)-carbazole-4-carboxamide; 9-benzyl-7-methoxy-5-((carboxamidomethyl)oxy)-carbazole-4-carboxamide; [9-Benzyl-4-carbamoyl-1,2,3,4-tetrahydrocarbazole-5-yl]oxyacetic acid; (R,S)-(9-benzyl-4-carbamoyl-1-oxo-3-thia-1,2,3,4-tetrahydrocarbazol-5-yl)oxyacetic acid; (R,S)-(9-benzyl-4-carbamoyl-3-thia-1,2,3,4-tetrahydrocarbazol-5-yl)oxyacetic acid; 2-(4-oxo-5-carboxamido-9-benzyl-9H-pyrido[3,4-b]indolyl)acetic acid chloride; [N-benzyl-1-carbamoyl-1-aza-1,2,3,4-tetrahydrocarbazol-8-yl]oxyacetic acid; 4-methoxy-6-methoxycarbonyl-10-phenylmethyl-6,7,8,9-tetrahydropyrido[1,2-a]indole; (4-carboxamido-9-phenylmethyl-4,5-dihydrothiopyrano[3,4-b]indol-5-yl)oxyacetic acid; 3,4-dihydro-4-carboxamidol-5-methoxy-9-phenylmethylpyrano[3,4-b]indole; 2-[(2,9 bis-benzyl-4-carbamoyl-1,2,3,4-tetrahydro-betacarbolin-5-yl)oxy]acetic acid; 2-[4-oxo-5-carboxamido-9-(2-methylbenzyl)-9H-pyrido[3,4-b]indolyl]acetic acid; 2-[4-oxo-5-carboxamido-9-(3-methylbenzyl)-9H-pyrido[3,4-b]indolyl]acetic acid; 2-[4-oxo-5-carboxamido-9-(4-methylbenzyl)-9H-pyrido[3,4-b]indolyl]acetic acid; 2-[4-oxo-5-carboxamido-9-(4-tert-butylbenzyl)-9H-pyrido[3,4-b]indolyl]acetic acid; 2-[4-oxo-5-carboxamido-9-pentafluorobenzyl-9H-pyrido[3,4-b]indolyl]acetic acid; 2-[4-oxo-5-carboxamido-9-(2-fluorobenzyl)-9H-pyrido[3,4-b]indolyl]acetic acid; 2-[4-oxo-5-carboxamido-9-(3-fluorobenzyl)-9H-pyrido[3,4-b]indolyl]acetic acid; 2-[4-oxo-5-carboxamido-9-(4-fluorobenzyl)-9H-pyrido[3,4-b]indolyl]acetic acid; 2-[4-oxo-5-carboxamido-9-(2,6-difluorobenzyl)-9H-pyrido[3,4-b]indolyl]acetic acid; 2-[4-oxo-5-carboxamido-9-(3,4-difluorobenzyl)-9H-pyrido[3,4-b]indolyl]acetic acid; 2-[4-oxo-5-carboxamido-9-(2,5-difluorobenzyl)-9H-pyrido[3,4-b]indolyl]acetic acid; 2-[4-oxo-5-carboxamido-9-(3,5-difluorobenzyl)-9H-pyrido[3,4-b]indolyl]acetic acid; 2-[4-oxo-5-carboxamido-9-(2,4-difluorobenzyl)-9H-pyrido[3,4-b]indolyl]acetic acid; 2-[4-oxo-5-carboxamido-9-(2,3-difluorobenzyl)-9H-pyrido[3,4-b]indolyl]acetic acid; 2-[4-oxo-5-carboxamido-9-[2-(trifluoromethyl)benzyl]-9H-pyrido[3,4-b]indolyl]acetic acid; 2-[4-oxo-5-carboxamido-9-[2-(trifluoromethyl)benzyl]-9H-pyrido[3,4-b]indolyl]acetic acid; 2-[4-oxo-5-carboxamido-9-[3-(trifluoromethyl)benzyl]-9H-pyrido[3,4-b]indolyl]acetic acid; 2-[4-oxo-5-carboxamido-9-[4-(trifluoromethyl)benzyl]-9H-pyrido[3,4-b]indolyl]acetic acid; 2-[4-oxo-5-carboxamido-9-[3,5-bis(trifluoromethyl)benzyl]-9H-pyrido[3,4-b]indolyl]acetic acid; 2-[4-oxo-5-carboxamido-9-[2,4-bis(trifluoromethyl)benzyl]-9H-pyrido[3,4-b]indolyl]acetic acid; 2-[4-oxo-5-carboxamido-9-(a-methylnaphthyl)-9H-pyrido[3,4-b]indolyl]acetic acid; 2-[4-oxo-5-carboxamido-9-(b-methylnaphthyl)-9H-pyrido[3,4-b]indolyl]acetic acid; 2-[4-oxo-5-carboxamido-9-(3,5-dimethylbenzyl)-9H-pyrido[3,4-b]indolyl]acetic acid; 2-[4-oxo-5-carboxamido-9-(2,4-dimethylbenzyl)-9H-pyrido[3,4-b]indolyl]acetic acid; 2-[4-oxo-5-carboxamido-9-(2-phenylbenzyl)-9H-pyrido[3,4-b]indolyl]acetic acid; 2-[4-oxo-5-carboxamido-9-(3-phenylbenzyl)-9H-pyrido[3,4-b]indolyl]acetic acid; 2-[4-oxo-5-carboxamido-9-(4-phenylbenzyl)-9H-pyrido[3,4-b]indolyl]acetic acid; 2-[4-oxo-5-carboxamido-9-(1-fluorenylmethy)-9H-pyrido[3,4-b]indolyl]acetic acid; 2-[4-oxo-5-carboxamido-9-(2-fluoro-3-methylbenzyl)-9H-pyrido[3,4-b]indolyl]acetic acid; 2-[4-oxo-5-carboxamido-9-(3-benzoylbenzyl)-9H-pyrido[3,4-b]indolyl]acetic acid; 2-[4-oxo-5-carboxamido-9-(2-phenoxybenzyl)-9H-pyrido[3,4-b]indolyl]acetic acid; 2-[4-oxo-5-carboxamido-9-(3-phenoxybenzyl)-9H-pyrido[3,4-b]indolyl]acetic acid; 2-[4-oxo-5-carboxamido-9-(4-phenoxybenzyl)-9H-pyrido[3,4-b]indolyl]acetic acid; 2-[4-oxo-5-carboxamido-9-[3-[2-(fluorophenoxy)benzyl]]-9H-pyrido[3,4-b]indolyl]acetic acid; 2-[4-oxo-5-carboxamido-9-[3-[4-(fluorophenoxy)benzyl]]-9H-pyrido[3,4-b]indolyl]acetic acid; 2-[4-oxo-5-carboxamido-9-[2-fluoro-3-(trifluoromethyl)benzyl]-9H-pyrido[3,4-b]indolyl]acetic acid; 2-[4-oxo-5-carboxamido-9-[2-fluoro-4-(trifluoromethyl)benzyl]-9H-pyrido[3,4-b]indolyl]acetic acid; 2-[4-oxo-5-carboxamido-9-[2-fluoro-5-(trifluoromethyl)benzyl]-9H-pyrido[3,4-b]indolyl]acetic acid; 2-[4-oxo-5-carboxamido-9-[3-fluoro-5-(trifluoromethyl)benzyl]-9H-pyrido[3,4-b]indolyl]acetic acid; 2-[4-oxo-5-carboxamido-9-[4-fluoro-2-(trifluoromethyl)benzyl]-9H-pyrido[3,4-b]indolyl]acetic acid; 2-[4-oxo-5-carboxamido-9-[4-fluoro-3-(trifluoromethyl)benzyl]-9H-pyrido[3,4-b]indolyl]acetic acid; 2-[4-oxo-5-carboxamido-9-[2-fluoro-6-(trifluoromethyl)benzyl]-9H-pyrido[3,4-b]indolyl]acetic acid; 2-[4-oxo-5-carboxamido-9-(2,3,6-trifluorobenzyl)-9H-pyrido[3,4-b]indolyl]acetic acid; 2-[4-oxo-5-carboxamido-9-(2,3,5-trifluorobenzyl)-9H-pyrido[3,4-b]indolyl]acetic acid; 2-[4-oxo-5-carboxamido-9-(2,4,5-trifluorobenzyl)-9H-pyrido[3,4-b]indolyl]acetic acid; 2-[4-oxo-5-carboxamido-9-(2,4,6-trifluorobenzyl)-9H-pyrido[3,4-b]indolyl]acetic acid; 2-[4-oxo-5-carboxamido-9-(2,3,4-trifluorobenzyl)-9H-pyrido[3,4-b]indolyl]acetic acid; 2-[4-oxo-5-carboxamido-9-(3,4,5-trifluorobenzyl)-9H-pyrido[3,4-b]indolyl]acetic acid; 2-[4-oxo-5-carboxamido-9-[3-(trifluoromethoxyl)benzyl]-9H-pyrido[3,4-b]indolyl]acetic acid; 2-[4-oxo-5-carboxamido-9-[4-(trifluoromethoxyl)benzyl]-9H-pyrido[3,4-b]indolyl]acetic acid; 2-[4-oxo-5-carboxamido-9-[4-methoxy(tetrafluoro)benzyl]-9H-pyrido[3,4-b]indolyl]acetic acid; 2-[4-oxo-5-carboxamido-9-(2-methoxybenzyl)-9H-pyrido[3,4-b]indolyl]acetic acid; 2-[4-oxo-5-carboxamido-9-(3-methoxybenzyl)-9H-pyrido[3,4-b]indolyl]acetic acid; 2-[4-oxo-5-carboxamido-9-(4-methoxybenzyl)-9H-pyrido[3,4-b]indolyl]acetic acid; 2-[4-oxo-5-carboxamido-9-(4-ethylbenzyl)-9H-pyrido[3,4-b]indolyl]acetic acid; 2-[4-oxo-5-carboxamido-9-(4-isopropylbenzyl)-9H-pyrido[3,4-b]indolyl]acetic acid; 2-[4-oxo-5-carboxamido-9-(3,4,5-trimethoxybenzyl)-9H-pyrido[3,4-b]indolyl]acetic acid; 2-[4-oxo-5-carboxamido-9-(3,4-methylenedioxybenzyl)-9H-pyrido[3,4-b]indolyl]acetic acid; 2-[4-oxo-5-carboxamido-9-(4-methoxy-3-methylbenzyl)-9H-pyrido[3,4-b]indolyl]acetic acid; 2-[4-oxo-5-carboxamido-9-(3,5-dimethoxybenzyl)-9H-pyrido[3,4-b]indolyl]acetic acid; 2-[4-oxo-5-carboxamido-9-(2,5-dimethoxybenzyl)-9H-pyrido[3,4-b]indolyl]acetic acid; 2-[4-oxo-5-carboxamido-9-(4-ethoxybenzyl)-9H-pyrido[3,4-b]indolyl]acetic acid; 2-[4-oxo-5-carboxamido-9-(cyclohexylmethyl)-9H-pyrido[3,4-b]indolyl]acetic acid; 2-[4-oxo-5-carboxamido-9-(cyclopentylmethyl)-9H-pyrido[3,4-b]indolyl]acetic acid; 2-[4-oxo-5-carboxamido-9-ethyl-9H-pyrido[3,4-b]indolyl]acetic acid; 2-[4-oxo-5-carboxamido-9-(1-propyl)-9H-pyrido[3,4-b]indolyl]acetic acid; 2-[4-oxo-5-carboxamido-9-(2-propyl)-9H-pyrido[3,4-b]indolyl]acetic acid; 2-[4-oxo-5-carboxamido-9-(1-butyl)-9H-pyrido[3,4-b]indolyl]acetic acid; 2-[4-oxo-5-carboxamido-9-(2-butyl)-9H-pyrido[3,4-b]indolyl]acetic acid; 2-[4-oxo-5-carboxamido-9-isobutyl-9H-pyrido[3,4-b]indolyl]acetic acid; 2-[4-oxo-5-carboxamido-9-[2-(1-phenylethyl)]-9H-pyrido[3,4-b]indolyl]acetic acid; 2-[4-oxo-5-carboxamido-9-[3-(1-phenylpropyl)]-9H-pyrido[3,4-b]indolyl]acetic acid; 2-[4-oxo-5-carboxamido-9-[4-(1-phenylbutyl)]-9H-pyrido[3,4-b]indolyl]acetic acid; 2-[4-oxo-5-carboxamido-9-(1-pentyl)-9H-pyrido[3,4-b]indolyl]acetic acid; 2-[4-oxo-5-carboxamido-9-(1-hexyl)-9H-pyrido[3,4-b]indolyl]acetic acid; 4-[(9-benzyl-4-carbamoyl-1,2,3,4-tetrahydrocarbazol-6-yl)oxy]butyric acid; 3-[(9-benzyl-4-carbamoyl-1,2,3,4-tetrahydrocarbazol-6-yl)oxy]propylphosphonic acid; 2-[(9-benzyl-4-carbamoyl-1,2,3,4-tetrahydrocarbazol-6-yl)oxy]methylbenzoic acid; 3-[(9-benzyl-4-carbamoyl-7-n-octyl-1,2,3,4-tetrahydrocarbazol-6-yl)oxy]propylphosphonic acid; 4-[(9-benzyl-4-carbamoyl-7-ethyl-1,2,3,4-tetrahydrocarbazol-6-yl)oxy]butyric acid; 3-[(9-benzyl-4-carbamoyl-7-ethyl-1,2,3,4-tetrahydrocarbazol-6-yl)oxy]propylphosphonic acid; 3-[(9-benzyl-4-carbamoyl-7-ethyl-1,2,3,4-tetrahydrocarbazol-6-yl)oxy]propylphosphonic acid; (S)-(+)-4-[(9-benzyl-4-carbamoyl-7-ethyl-1,2,3,4-tetrahydrocarbazol-6-yl)oxy]butyric acid; 4-[9-benzyl-4-carbamoyl-6-(2-cyanoethyl)-1,2,3,4-tetrahydrocarbazol-6-yl]oxybutyric acid; 4-[9-benzyl-4-carboxamido-7-(2-phenylethyl)-1,2,3,4-tetrahydrocarbazol-6-yl]oxybutyric acid; 4-[9-benzyl-4-carboxamidocarbazol-6-yl]oxybutyric acid; methyl 2-[(9-benzyl-4-carbamoyl-1,2,3,4-tetrahydrocarbazol-6-yl)oxy]methylbenzoate; 4-[9-benzyl-4-carbamoyl-7-(2-cyanoethyl)-1,2,3,4-tetrahydrocarbazol-6-yl]oxybutyric acid; 9-benzyl-7-methoxy-5-cyanomethyloxy-1,2,3,4-tetrahydrocarbazole-4-carboxamide; [9-benzyl-4-carbamoyl-8-methyl-carbazole-5-yl]oxyacetic acid; and [9-benzyl-4-carbamoyl-carbazole-5-yl]oxyacetic acid, or pharmaceutically acceptable salts, solvates, prodrug derivatives, racemates, tautomers, or optical isomers thereof.

Certain embodiments of the methods, compositions, and kits provided herein utilize the sPLA₂ inhibitor 3-(2-Amino-1,2-dioxoethyl)-2-ethyl-1-(phenylmethyl)-1H-indol-4-yl)oxy)acetic acid (A-001, also referred to in the art as S-5920 or LY315920) or a salt, solvate, polymorph, or co-crystal thereof. A-001 has the structure:

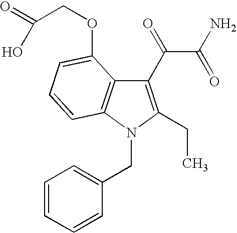

A-001 is a competitive inhibitor of sPLA₂. Certain embodiments of the methods, compositions, and kits provided herein utilize the sodium salt of A-001.

Certain embodiments of the methods, compositions, and kits provided herein utilize [[3-(2-Amino-1,2-dioxoethyl)-2-ethyl-1-(phenylmethyl)-1H-indol-4-yl]oxy]acetic acid methyl ester (A-002, also referred to in the art as S-3013, LY333013, or varespladib methyl), which has the structure:

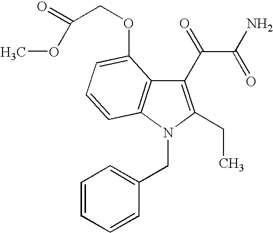

A-002 is a prodrug of A-001 that is rapidly absorbed and hydrolyzed to the active A-001 molecule. A-002 has a terminal half-life (t_{1/2}) of approximately ten hours.

In certain embodiments, the methods and compositions provided herein may utilize other prodrug forms of A-001 besides A-002, including one or more C₁-C₆ alkyl esters, acyloxyalkyl esters, or alkyloxycarbonyloxyalkyl esters of A-001. Since each of these prodrugs is hydrolyzed to the same active molecule, one of ordinary skill in the art would expect them to have similar therapeutic characteristics, and such a skilled artisan could identify such prodrugs with minimal experimentation.

As with statins, the synergistic effect between sPLA₂ inhibitors and niacin drugs was not limited to one particular type of niacin drug. Instead, the effect was observed across a range of niacin drugs that included niacin, nicotinic acid, acipimox, ERN (Niaspan®), Niaspan® in combination with lovastatin (Advicor®), and Niaspan® in combination with simvastatin (Simcor®). Other niacin drugs that can be utilized in the methods, compositions, and kits provided herein include niacin derivatives and formulations such as xanthinol niacinate, 1-methylnicotinamide, 1-methyl-N′-hydroxymethylnicotinamide, niacin immediate-release (Niacor®), ERN derivatives such as Niaspan MF or Niaspan CF, and ERN combinations such as ERN plus the DP-1 antagonist laropiprant (MK-0524A, marketed as Cordaptive® and Tredaptive®).

Since the synergistic effect of A-002 and niacin drugs was generalized across the entire class of niacin drugs, one skilled in the art would expect to obtain similar results by combining sPLA₂ inhibitors with drugs that have a similar mechanism of action to niacin. Therefore, in certain embodiments, the methods, compositions, and kits disclosed herein may utilize compounds with mechanisms similar to niacin in lieu of one or more niacin drugs, including for example inhibitors of diacylglycerol acyltransferase-2 (DGAT2), agonists of niacin receptors such as GPR109A and GP109B, and compounds that inhibit HDL-ApoA1 uptake or removal.

Nearly all of the subjects receiving niacin drugs in the clinical studies described herein were also receiving one or more statins before and during the trial period. Therefore, provided herein are compositions, methods, and kits for treating CVD, decreasing TG levels, increasing HDL levels, and increasing HDL/LDL ratios using a combination of one or more sPLA₂ inhibitors, one or more niacin drugs, and one or more statins.

In those embodiments of the compositions and methods disclosed herein that utilize statins, examples of statins that may be used include, but are not limited to, atorvastatin or atorvastatin calcium (marketed as Lipitor® or Torvast®; see, e.g., U.S. Pat. No. 4,681,893 or 5,273,995) and atorvastatin combinations (e.g., atorvastatin plus amlodipine (marketed as Norvasc®), combination marketed as Caduet®, see, e.g., U.S. Pat. No. 6,455,574; atorvastatin plus CP-529414 (marketed as Torcetrapib®); atorvastatin plus APA-01; atorvastatin plus ezetimibe), cerivastatin (marketed as Lipobay® or Baycol®), fluvastatin (marketed as Lescol®; U.S. Pat. No. 4,739,073), lovastatin (marketed as Mevacor® or Altocor®; see, e.g., U.S. Pat. No. 4,231,938), lovastatin combinations (e.g., lovastatin plus Niaspan®, combination marketed as Advicor®), mevastatin, pitavastatin (marketed as Livalo® or Pitava®), pravastatin (marketed as Pravachol®, Mevalotin®, Selektine®, or Lipostat®; see, e.g., U.S. Pat. No. 4,346,227), pravastatin combinations (e.g., pravastatin plus fenofibrate), rosuvastatin (marketed as Crestor®), rosuvastatin combinations (e.g., rosuvastatin plus TriCor®), simvastatin (marketed as Zocor® or Lipex®; see, e.g., U.S. Pat. Nos. 4,444,784; 4,916,239; and 4,820,850), and simvastatin combinations (e.g., simvastatin plus ezetimibe, combination marketed as Vytorin®, see, e.g., U.S. Pat. No. 7,229,982; simvastatin plus Niaspan®, combination marketed as Simcor®; simvastatin plus MK-0524A, combination referred to as MK-0524B), as well as various pharmaceutically acceptable salts, solvates, salts, stereoisomers, prodrugs derivatives, or nitroderivatives of the compounds listed above. In some cases, such as for example with simvastatin, the active form of the statin is a metabolite formed in the body of a subject following administration. In other cases, statins are administered in their active form. In certain embodiments, statins may be administered according to their standard recommended dosage, while in other embodiments statins may be administered lower than the recommended dosage.

In certain embodiments, compositions are provided comprising one or more sPLA₂ inhibitors and one or more niacin drugs. In certain embodiments, the one or more sPLA₂ inhibitors comprise A-001 or a pharmaceutically acceptable salt, solvate, polymorph, co-crystal, or prodrug thereof, and in certain of these embodiments, the prodrug thereof is a C₁-C₆ alkyl ester, acyloxyalkyl ester, or alkyloxycarbonyloxyalkyl ester. In certain of these embodiments, the prodrug is A-002. In certain embodiments, the niacin drug is niacin, immediate-release niacin, nicotinic acid, acipimox, ERN, ERN in combination with lovastatin, ERN in combination with simvastatin, ERN in combination with laropiprant, xanthinol niacinate, 1-methylnicotinamide, or 1-methyl-N′-hydroxymethylnicotinamide. In certain embodiments, the compositions provided herein may comprise one or more pharmaceutically acceptable carriers. In certain embodiments, the compositions provided herein further comprise one or more statins. In these embodiments, the one or more statins may be atorvastatin, cerivastatin, fluvastatin, lovastatin, mevastatin, pitavastatin, pravastatin, rosuvastatin, simvastatin, or a statin combination drug such as atorvastatin plus ezetimibe, atorvastatin plus amlodipine, atorvastatin plus CP-529414, atorvastatin plus APA-01, simvastatin plus ezetimibe, simvastatin plus extended release niacin, simvastatin plus MK-0524A, lovastatin plus extended release niacin, rosuvastatin plus fenofibrate, pravastatin plus fenofibrate, or statin plus TAK-457, or a pharmaceutically acceptable salt, solvate, salt, stereoisomer, prodrug derivative, or nitroderivative thereof.

In certain embodiments, methods are provided for treating CVD in a subject in need thereof by administering one or more sPLA₂ inhibitors in combination with one or more niacin drugs. In certain embodiments, the one or more sPLA₂ inhibitors comprise A-001 or a pharmaceutically acceptable salt, solvate, polymorph, co-crystal, or prodrug thereof, and in certain of these embodiments, the prodrug of A-001 is a C₁-C₆ alkyl ester, acyloxyalkyl ester, or alkyloxycarbonyloxyalkyl ester. In certain of these embodiments, the prodrug is A-002. In certain embodiments, the one or more niacin drugs comprise niacin, immediate-release niacin, nicotinic acid, acipimox, ERN, ERN in combination with lovastatin, ERN in combination with simvastatin, ERN in combination with laropiprant, xanthinol niacinate, 1-methylnicotinamide, or 1-methyl-N′-hydroxymethylnicotinamide. In certain embodiments, these methods further comprise administering one or more statins. In those embodiments wherein one or more statins are administered, the one or more statins may be atorvastatin, cerivastatin, fluvastatin, lovastatin, mevastatin, pitavastatin, pravastatin, rosuvastatin, simvastatin, or a statin combination drug such as atorvastatin plus ezetimibe, atorvastatin plus amlodipine, atorvastatin plus CP-529414, atorvastatin plus APA-01, simvastatin plus ezetimibe, simvastatin plus extended release niacin, simvastatin plus MK-0524A, lovastatin plus extended release niacin, rosuvastatin plus fenofibrate, pravastatin plus fenofibrate, or statin plus TAK-457, or a pharmaceutically acceptable salt, solvate, salt, stereoisomer, prodrug derivative, or nitroderivative thereof. In certain embodiments, the CVD being treated is CAD, CHD, or a condition associated with CAD or CHD, and in certain of these embodiments the CVD or condition associated therewith is ACS or dyslipidemia. In certain embodiments, the subject being treated has experienced a major adverse cardiac event (i.e., cardiovascular death, fatal and non-fatal MI, documented UA requiring urgent hospitalization, need for revascularization 60 days or more after an ACS event, or fatal or non-fatal stroke), has been deemed at risk of experiencing a major adverse cardiac event, or has exhibited one or more symptoms associated with a major adverse cardiac event.

In certain embodiments, methods are provided for decreasing TG levels and/or increasing HDL levels in a subject in need thereof by administering one or more sPLA₂ inhibitors in combination with one or more niacin drugs. In certain embodiments, the one or more sPLA₂ inhibitors comprise A-001 or a pharmaceutically acceptable salt, solvate, polymorph, co-crystal, or prodrug thereof, and in certain of these embodiments, the prodrug of A-001 is a C₁-C₆ alkyl ester, acyloxyalkyl ester, or alkyloxycarbonyloxyalkyl ester. In certain embodiments, the prodrug is A-002. In certain embodiments, the one or more niacin drugs comprise niacin, immediate-release niacin, nicotinic acid, acipimox, ERN, ERN in combination with lovastatin, ERN in combination with simvastatin, ERN in combination with laropiprant, xanthinol niacinate, 1-methylnicotinamide, or 1-methyl-N′-hydroxymethylnicotinamide. In certain embodiments, co-administration of one or more sPLA₂ inhibitors and one or more niacin drugs results in a decrease in TG levels, an increase in HDL levels, or a combination thereof. In certain of these embodiments, the decrease in TG levels and/or the increase in HDL levels is greater than would be expected from a simple additive effect between the one or more sPLA₂ inhibitors and the one or more niacin drugs. In certain embodiments, these methods further comprise administering one or more statins. In those embodiments wherein one or more statins are administered, the one or more statins may be atorvastatin, cerivastatin, fluvastatin, lovastatin, mevastatin, pitavastatin, pravastatin, rosuvastatin, simvastatin, or a statin combination drug such as atorvastatin plus ezetimibe, atorvastatin plus amlodipine, atorvastatin plus CP-529414, atorvastatin plus APA-01, simvastatin plus ezetimibe, simvastatin plus extended release niacin, simvastatin plus MK-0524A, lovastatin plus extended release niacin, rosuvastatin plus fenofibrate, pravastatin plus fenofibrate, or statin plus TAK-457, or a pharmaceutically acceptable salt, solvate, salt, stereoisomer, prodrug derivative, or nitroderivative thereof.

In certain embodiments, methods are provided for decreasing TG levels and/or increasing HDL levels to a pre-determined target level a subject in need thereof by administering one or more sPLA₂ inhibitors in combination with one or more niacin drugs. In certain embodiments, the one or more sPLA₂ inhibitors comprise A-001 or a pharmaceutically acceptable salt, solvate, polymorph, co-crystal, or prodrug thereof, and in certain of these embodiments, the prodrug of A-001 is a C₁-C₆ alkyl ester, acyloxyalkyl ester, or alkyloxycarbonyloxyalkyl ester. In certain embodiments, the prodrug is A-002. In certain embodiments, the one or more niacin drugs comprise niacin, immediate-release niacin, nicotinic acid, acipimox, ERN, ERN in combination with lovastatin, ERN in combination with simvastatin, ERN in combination with laropiprant, xanthinol niacinate, 1-methylnicotinamide, or 1-methyl-N′-hydroxymethylnicotinamide. In certain embodiments, these methods further comprise administering one or more statins. In those embodiments wherein one or more statins are administered, the one or more statins may be atorvastatin, cerivastatin, fluvastatin, lovastatin, mevastatin, pitavastatin, pravastatin, rosuvastatin, simvastatin, or a statin combination drug such as atorvastatin plus ezetimibe, atorvastatin plus amlodipine, atorvastatin plus CP-529414, atorvastatin plus APA-01, simvastatin plus ezetimibe, simvastatin plus extended release niacin, simvastatin plus MK-0524A, lovastatin plus extended release niacin, rosuvastatin plus fenofibrate, pravastatin plus fenofibrate, or statin plus TAK-457, or a pharmaceutically acceptable salt, solvate, salt, stereoisomer, prodrug derivative, or nitroderivative thereof.

In certain embodiments, methods are provided for treating dyslipidemia in a subject in need thereof by administering one or more sPLA₂ inhibitors in combination with one or more niacin drugs. In certain embodiments, the one or more sPLA₂ inhibitors comprise A-001 or a pharmaceutically acceptable salt, solvate, polymorph, co-crystal, or prodrug thereof, and in certain of these embodiments, the prodrug of A-001 is a C₁-C₆ alkyl ester, acyloxyalkyl ester, or alkyloxycarbonyloxyalkyl ester. In certain of these embodiments, the prodrug is A-002. In certain embodiments, the one or more niacin drugs comprise niacin, immediate-release niacin, nicotinic acid, acipimox, ERN, ERN in combination with lovastatin, ERN in combination with simvastatin, ERN in combination with laropiprant, xanthinol niacinate, 1-methylnicotinamide, or 1-methyl-N′-hydroxymethylnicotinamide. In certain embodiments, these methods further comprise administering one or more statins. In those embodiments wherein one or more statins are administered, the one or more statins may be atorvastatin, cerivastatin, fluvastatin, lovastatin, mevastatin, pitavastatin, pravastatin, rosuvastatin, simvastatin, or a statin combination drug such as atorvastatin plus ezetimibe, atorvastatin plus amlodipine, atorvastatin plus CP-529414, atorvastatin plus APA-01, simvastatin plus ezetimibe, simvastatin plus extended release niacin, simvastatin plus MK-0524A, lovastatin plus extended release niacin, rosuvastatin plus fenofibrate, pravastatin plus fenofibrate, or statin plus TAK-457, or a pharmaceutically acceptable salt, solvate, salt, stereoisomer, prodrug derivative, or nitroderivative thereof.

In certain embodiments, methods are provided for improving HDL/LDL ratio in a subject in need thereof by administering one or more sPLA₂ inhibitors in combination with one or more niacin drugs. In certain embodiments, the one or more sPLA₂ inhibitors comprise A-001 or a pharmaceutically acceptable salt, solvate, polymorph, co-crystal, or prodrug thereof, and in certain of these embodiments, the prodrug of A-001 is a C₁-C₆ alkyl ester, acyloxyalkyl ester, or alkyloxycarbonyloxyalkyl ester. In certain of these embodiments, the prodrug is A-002. In certain embodiments, the one or more niacin drugs comprise niacin, immediate-release niacin, nicotinic acid, acipimox, ERN, ERN in combination with lovastatin, ERN in combination with simvastatin, ERN in combination with laropiprant, xanthinol niacinate, 1-methylnicotinamide, or 1-methyl-N′-hydroxymethylnicotinamide. In certain embodiments, the improvement in HDL/LDL ratios is accomplished at least in part by increasing HDL levels. In certain embodiments, these methods further comprise administering one or more statins. In those embodiments wherein one or more statins are administered, the one or more statins may be atorvastatin, cerivastatin, fluvastatin, lovastatin, mevastatin, pitavastatin, pravastatin, rosuvastatin, simvastatin, or a statin combination drug such as atorvastatin plus ezetimibe, atorvastatin plus amlodipine, atorvastatin plus CP-529414, atorvastatin plus APA-01, simvastatin plus ezetimibe, simvastatin plus extended release niacin, simvastatin plus MK-0524A, lovastatin plus extended release niacin, rosuvastatin plus fenofibrate, pravastatin plus fenofibrate, or statin plus TAK-457, or a pharmaceutically acceptable salt, solvate, salt, stereoisomer, prodrug derivative, or nitroderivative thereof.

In certain embodiments, methods are provided for increasing HDL/LDL ratio to a pre-determined target level in a subject in need thereof by administering one or more sPLA₂ inhibitors in combination with one or more niacin drugs. In certain embodiments, the one or more sPLA₂ inhibitors comprise A-001 or a pharmaceutically acceptable salt, solvate, polymorph, co-crystal, or prodrug thereof, and in certain of these embodiments, the prodrug of A-001 is a C₁-C₆ alkyl ester, acyloxyalkyl ester, or alkyloxycarbonyloxyalkyl ester. In certain of these embodiments, the prodrug is A-002. In certain embodiments, the one or more niacin drugs comprise niacin, immediate-release niacin, nicotinic acid, acipimox, ERN, ERN in combination with lovastatin, ERN in combination with simvastatin, ERN in combination with laropiprant, xanthinol niacinate, 1-methylnicotinamide, or 1-methyl-N′-hydroxymethylnicotinamide. In certain embodiments, the increase in HDL/LDL ratio to a target level is accomplished at least in part by increasing HDL levels. In certain embodiments, these methods further comprise administering one or more statins. In those embodiments wherein one or more statins are administered, the one or more statins may be atorvastatin, cerivastatin, fluvastatin, lovastatin, mevastatin, pitavastatin, pravastatin, rosuvastatin, simvastatin, or a statin combination drug such as atorvastatin plus ezetimibe, atorvastatin plus amlodipine, atorvastatin plus CP-529414, atorvastatin plus APA-01, simvastatin plus ezetimibe, simvastatin plus extended release niacin, simvastatin plus MK-0524A, lovastatin plus extended release niacin, rosuvastatin plus fenofibrate, pravastatin plus fenofibrate, or statin plus TAK-457, or a pharmaceutically acceptable salt, solvate, salt, stereoisomer, prodrug derivative, or nitroderivative thereof.

In certain embodiments, methods are provided for increasing the effectiveness of one or more niacin drugs by administering one or more sPLA₂ inhibitors. In certain embodiments, the one or more sPLA₂ inhibitors comprise A-001 or a pharmaceutically acceptable salt, solvate, polymorph, co-crystal, or prodrug thereof, and in certain of these embodiments, the prodrug of A-001 is a C₁-C₆ alkyl ester, acyloxyalkyl ester, or alkyloxycarbonyloxyalkyl ester. In certain embodiments, the prodrug is A-002. An increase in effectiveness of a niacin drug may refer to an increase in the therapeutic effect of the niacin drug, a decrease in the dosage of the niacin drug required to obtain a particular level of therapeutic effect, or some combination thereof. As discussed above, niacin administration is associated with a variety of deleterious side effects. Therefore, methods that decrease the dosage of a niacin drug necessary to obtain a desired therapeutic effect are attractive because decreased niacin dosages are likely to result in decreased niacin-related side effects.

In certain embodiments, kits are provided that comprise one or more sPLA₂ inhibitors and one or more niacin drugs. In certain embodiments, the one or more sPLA₂ inhibitors comprise A-001 or a pharmaceutically acceptable salt, solvate, polymorph, co-crystal, or prodrug thereof, and in certain of these embodiments, the prodrug of A-001 is a C₁-C₆ alkyl ester, acyloxyalkyl ester, or alkyloxycarbonyloxyalkyl ester. In certain embodiments, the prodrug is A-002. In certain embodiments, the one or more niacin drugs comprise niacin, immediate-release niacin, nicotinic acid, acipimox, ERN, ERN in combination with lovastatin, ERN in combination with simvastatin, ERN in combination with laropiprant, xanthinol niacinate, 1-methylnicotinamide, or 1-methyl-N′-hydroxymethylnicotinamide. In certain embodiments, the kits further provide one or more statins. In certain embodiments, the kits provided herein include instructions for usage, such as dosage or administration instructions. In certain of these embodiments, the instructions may describe the administration of one or more compounds included in the kit for the treatment of CVD, lowering TG, increasing HDL, and/or improving HDL/LDL ratio. Within the kit, the one or more sPLA₂ inhibitors, the one or more niacin drugs, and/or the one or more statins may be divided into separate compartments. For example, the kit may comprise multiple bottles or packets, wherein each bottle or packet contains either one or more sPLA₂ inhibitors or one or more niacin drugs. In other embodiments, one or more sPLA₂ inhibitors and the one or more niacin drugs may be found in a single, undivided container.

In certain embodiments, the use of one or more sPLA₂ inhibitors and one or more niacin drugs for preparation or manufacture of a medicament for treating CVD, decreasing TG levels, increasing HDL levels, and increasing HDL/LDL ratio is provided. In certain embodiments, the one or more sPLA₂ inhibitors comprise A-001 or a pharmaceutically acceptable salt, solvate, polymorph, co-crystal, or prodrug thereof, and in certain of these embodiments, the prodrug of A-001 is a C₁-C₆ alkyl ester, acyloxyalkyl ester, or alkyloxycarbonyloxyalkyl ester. In certain embodiments, the prodrug is A-002. In certain embodiments, the one or more niacin drugs comprise niacin, immediate-release niacin, nicotinic acid, acipimox, ERN, ERN in combination with lovastatin, ERN in combination with simvastatin, ERN in combination with laropiprant, xanthinol niacinate, 1-methylnicotinamide, or 1-methyl-N′-hydroxymethylnicotinamide.

In certain embodiments of the methods provided herein, one or more sPLA₂ inhibitors and one or more niacin drugs may be administered to a subject in a single composition. Provided herein in certain embodiments are these compositions, as well as kits comprising these compositions and the use of one or more sPLA₂ inhibitors and one or more niacin drugs in producing these compositions. In these embodiments, the composition may be administered to a subject on a one-time basis or in multiple administrations. In those embodiments wherein the composition is given in multiple administrations, it may be administered at set intervals over a particular time period determined in advance, or it may be administered indefinitely or until a particular therapeutic benchmark is reached, such as for example until a subject exhibits TG levels below a specified threshold, HDL levels above a certain threshold, or HDL/LDL ratios above a certain threshold. In certain embodiments, the composition may be administered from once or more times per day to once every month or once every several months. In certain of these embodiments, the composition is administered twice a day, and in certain other embodiments the composition is administered once a day.

In those embodiments wherein one or more statins are administered to a subject in conjunction with one or more sPLA₂ inhibitors and one or more niacin drugs, and wherein one or more sPLA₂ inhibitors and one or more niacin drugs are administered in a single composition, the one or more statins may be administered separately from the other compounds. Alternatively, the one or more statins may be administered as part of the sPLA₂ inhibitor/niacin composition. In those embodiments wherein the one or more statins are administered separately from the sPLA₂ inhibitor/niacin composition, the one or more statins may be administered simultaneously or sequentially with the sPLA₂ inhibitor/niacin composition, or they may be administered at a different time. The one or more statins may be administered more or less frequently than the sPLA₂ inhibitor/niacin composition. In certain embodiments, the one or more statins may be administered based on the standard dosage schedule as known in the art.

In certain embodiments of the methods provided herein, one or more sPLA₂ inhibitors and one or more niacin drugs may each be administered to a subject separately, i.e., in two or more separate compositions. In these embodiments, the one or more sPLA₂ inhibitors and the one or more niacin drugs may be administered simultaneously or sequentially. Further, the one or more sPLA₂ inhibitors and the one or more niacin drugs may be administered at different times and via different routes, and one compound may be administered more or less frequently than another. In certain embodiments, each of the compounds may be administered anywhere from once or more times per day to once every week, once every month, or once every several months. In certain embodiments, the one or more sPLA₂ inhibitors may be administered twice a day, and in other embodiments the one or more sPLA₂ inhibitors may be administered once a day. In certain embodiments, the one or more niacin drugs may be administered based on the standard dosage schedule for those drugs as known in the art. In certain embodiments, one or more sPLA₂ inhibitors and/or one or more niacin drugs may be administered to a subject on a one-time basis or in multiple administrations. In those embodiments wherein one or more of these compounds are given in multiple administrations, they may be administered at set intervals over a particular time period determined in advance, or they may be administered indefinitely or until a particular therapeutic benchmark is reached, such as for example until a subject exhibits TG levels below a specified threshold, HDL levels above a certain threshold, or HDL/LDL ratios above a certain threshold. In certain embodiments, administration of one or more sPLA₂ inhibitors and one or more niacin drugs may begin at the same time. In other embodiments, administration of one or more sPLA₂ inhibitors and one or more niacin drugs may begin at different times.

In those embodiments wherein one or more statins are administered to a subject in conjunction with one or more sPLA₂ inhibitors and one or more niacin drugs, the one or more statins may be administered separately from the other compounds. In certain of these embodiments, the one or more statins may be administered in a separate composition from both the one or more sPLA₂ inhibitors and the one or more niacin drugs. In other embodiments, the one or more statins may be administered in the same composition as either the one or more sPLA₂ inhibitors or the one or more of the niacin drugs. In these embodiments, the one or more sPLA₂ inhibitors, the one or more niacin drugs, and the one or more statins may be administered simultaneously or sequentially, or they may be administered at different times. Further, one or more of the compounds may be administered more or less frequently than the other compounds. In certain embodiments, the one or more statins may be administered based on the standard dosage schedule as known in the art.

Pharmaceutical compositions comprising one or more sPLA₂ inhibitors, one or more niacin drugs, and/or one or more statins may be administered by any administration pathway known in the art, including but not limited to oral, aerosol, enteral, nasal, ophthalmic, parenteral, or transdermal (e.g., topical cream, gel, lotion, or ointment, patch). “Parenteral” refers to a route of administration that is generally associated with injection, including infraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. One or more sPLA₂ inhibitors, one or more niacin drugs, and/or one or more statins may be administered in any pharmaceutically acceptable form, including for example in the form of a solid, liquid solution, suspension, emulsion, dispersion, micelle, or liposome. Preparations for injection may include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile emulsions. The solutions may be either aqueous or nonaqueous. In certain embodiments, the compositions may comprise one or more pharmaceutically acceptable carriers or may be administered in conjunction with one or more pharmaceutically acceptable carriers.

In certain embodiments, pharmaceutical compositions comprising one or more sPLA₂ inhibitors, one or more niacin drugs, and/or one or more statins may be formed into dosage units for ease of administration and uniformity of dosage. In certain of these embodiments, the dosage units may be oral dosage units, such as for example tablets, pills, or capsules. These oral dosage units may comprise the active ingredients (e.g., one or more sPLA₂ inhibitors, one or more niacin drugs) and one or more pharmaceutically acceptable carriers. In certain embodiments, pharmaceutical compositions comprising one or more sPLA₂ inhibitors, one or more niacin drugs, and/or one or more statins may be administered via a time release delivery vehicle, such as for example a time release oral dosage unit. A “time release vehicle” as used herein refers to any delivery vehicle that releases active agent (e.g., one or more sPLA₂ inhibitors, one or more niacin drugs, one or more sPLA₂ inhibitors plus one or more niacin drugs) at some time after administration or over a period of time following administration rather than immediately upon administration. Time release may be obtained by a coating on the vehicle that dissolves over a set timeframe following administration. In certain embodiments, the time release vehicle may comprise multiple layers of coating alternated with multiple layers of active ingredients, such that each layer of coating releases a certain volume of active ingredients as it dissolves. In other embodiments, one or more sPLA₂ inhibitors, one or more niacin drugs, and/or one or more statins may be administered via an immediate release delivery vehicle.

A therapeutically effective amount of one or more sPLA₂ inhibitors, one or more niacin drugs, or one or more statins may be determined for each compound individually. For example, niacin drugs may be administered or included in a pharmaceutical composition at a dosage that is known in the art to decrease TG levels and/or increase HDL levels. One skilled in the art will recognize that in those embodiments wherein one or more niacin drugs are combined with one or more sPLA₂ inhibitors in a single composition, the amount of niacin drug that constitutes a therapeutically effective amount may be different than the amount of niacin drug that constitutes a therapeutically effective amount when administered alone due to, for example, interactions between the niacin drug and the one or more sPLA₂ inhibitors. For example, the effective dosage of a niacin drug for use in combination therapy may be lower than the effective dosage for the niacin drug when administered alone. Likewise, the therapeutically effective amount of one or more sPLA₂ inhibitors may be lower when administered in conjunction with a niacin drug than when one or more sPLA₂ inhibitors are administered alone. In these situations, one skilled in the art will readily be able to determine a therapeutically effective amount for the combination using methods well known in the art. One skilled in the art will recognize that the therapeutically effective amount of one or more sPLA₂ inhibitors may vary within the compositions, methods, and kits disclosed herein. In certain embodiments, a therapeutically effective amount of one or more sPLA₂ inhibitors for use in combination with one or more niacin drugs is about 25 to about 5,000 mg/dose, and in certain of these embodiments a therapeutically effective amount may be from about 50 to about 500 mg/dose. In certain embodiments, a therapeutically effective amount of one or more niacin drug for use in combination with one or more sPLA₂ inhibitors is about 500 to about 3,000 mg/dose, and in certain of these embodiments a therapeutically effective amount may be from about 1,000 to about 2,000 mg/dose.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1

### Combined Effect of A-002 and Niacin on TG and HDL Levels in Human Subjects with Stable CAD

As previously disclosed, once or twice a day oral administration of A-002 to 361 human subjects with stable CAD at total daily dosages of 100 mg, 200 mg, 250 mg, 500 mg, or 1000 mg resulted in a decrease in serum LDL, LDL particle, small LDL particle, oxidized LDL, total cholesterol, ApoB, TG, sPLA₂, CRP, and IL-6 levels at all dosages tested. Reductions in LDL levels were observed not only in the ITT population, but also in subpopulations with baseline LDL levels at or above 72.0 mg/dl, subpopulations with diabetes, and subpopulations receiving statin, ezetimibe, and other compounds used in the treatment of CVD during the trial. Importantly, administration of A-002 in conjunction with statin or ezetimibe resulted in a decrease in LDL and small LDL particle levels greater than the expected additive decrease, indicating that A-002 lowers LDL and LDL particle levels in a synergistic manner when administered in conjunction with one or more statins and/or ezetimibe.

The data from the above studies was further examined to determine whether co-administration of A-002 with various niacin drugs had a similar synergistic effect on lipid levels. 46 subjects from the above once-a-day and twice-a-day A-002 studies were identified as receiving niacin drugs before and during the trial period. 45 of these 46 subjects were also receiving one or more statins before and during the trial period. The niacin drugs included niacin, nicotinic acid, acipimox, ERN (Niaspan®), Niaspan® in combination with lovastatin (Advicor®), and/or Niaspan® in combination with simvastatin (Simcor®). Of the 46 niacin subjects, twelve were concomitantly administered placebo rather than A-002 (i.e., niacin drug only), four were administered 100 mg of A-002 daily, five were administered 200 mg of A-002 daily, five were administered 250 mg of A-002 daily, thirteen were administered 500 mg of A-002 daily, and seven were administered 1000 mg of A-002 daily. In addition, 327 subjects were identified from the earlier studies that had received A-002 at any dosage but no niacin, and 103 subjects were identified that had received neither drug.

The change from baseline HDL and TG levels at eight weeks was determined for each of the subjects receiving niacin drugs, and the percentage increase or decrease in these levels was determined using an analysis of covariance (ANCOVA) model that calculated subject-specific percent change from baseline. Subjects receiving niacin drugs were not expected to exhibit an increase in HDL levels or a decrease in TG levels because these subjects had been on niacin drugs prior to the start of the trial, and therefore had already reached a steady-state with regard to the effect of niacin drugs on HDL and TG levels.

Subjects receiving placebo only, A-002 only, or niacin drugs only exhibited a decrease in mean HDL levels versus baseline after eight weeks. Subjects receiving niacin drugs concomitantly with A-002, on the other hand, exhibited a 3.1% increase in mean HDL levels versus baseline, and a 5.4% increase versus placebo. HDL results are summarized in Table 1.

TABLE 1
Change in baseline HDL levels

| | Baseline | Week 8 | Change | % change | Difference |
|---|---|---|---|---|---|
| | Mean | Mean | from | versus | versus |
| | HDL | HDL | baseline | baseline | placebo |
| Placebo only | 48.80 mg/dl | 47.44 mg/dl | −1.36 mg/dl | −2.3% | — |
| (n = 103) | | | | | |
| A-002 only (all | 47.80 mg/dl | 46.28 mg/dl | −1.52 mg/dl | −2.0% | +0.3% |
| dosages) | | | | | |
| (n = 327) | | | | | |
| Niacin only (n = 12) | 50.67 mg/dl | 46.75 mg/dl | −3.92 mg/dl | −6.2% | −3.9% |
| A-002 (all | 49.24 mg/dl | 50.35 mg/dl | +1.11 mg/dl | +3.1% | +5.4% |
| dosages) | | | | | |
| plus niacin | | | | | |
| (n = 34) | | | | | |

Administration of A-002 alone resulted in a 2.0% decrease in HDL versus baseline and a 0.3% increase in HDL versus placebo, while administration of niacin drugs alone resulted in a 6.2% decrease in HDL versus baseline and a 3.9% decrease in HDL versus placebo. If A-002 and niacin drugs had a simple additive effect on HDL levels, administration of a combination of A-002 and niacin drugs would be expected to decrease HDL levels by 8.2% versus baseline (−2.0% plus −6.2%), and by 3.6% versus placebo (0.3% plus −3.9%). However, the combined effect of A-002 and niacin drug administration was instead a 3.1% increase in HDL levels versus baseline and a 5.4% increase in HDL levels versus placebo, indicating that A-002 and niacin drugs unexpectedly increase HDL levels in a synergistic manner.

Subjects receiving placebo only, A-002 only, or niacin drugs only exhibited an increase in mean TG levels versus baseline after eight weeks. Subjects receiving niacin drugs concomitantly with A-002, on the other hand, exhibited an 8.1% decrease in mean TG levels versus baseline, and a 14.4% decrease versus placebo. TG results are summarized in Table 2.

TABLE 2
Change in baseline TG levels

| | | | | % change | Difference |
|---|---|---|---|---|---|
| | Baseline | Week 8 | Change from | versus | versus |
| | Mean TG | Mean TG | baseline | baseline | placebo |
| Placebo | 155.44 mg/dl | 156.65 mg/dl | +1.21 mg/dl | +6.3% | — |
| only (n = 103) | | | | | |
| A-002 only | 165.83 mg/dl | 159.04 mg/dl | −6.79 mg/dl | −3.7% | −2.6% |
| (n = 327) | | | | | |
| Niacin only | 129.25 mg/dl | 133.42 mg/dl | +4.17 mg/dl | +9.7% | +3.4% |
| (n = 12) | | | | | |
| A-002 + | 147.5 mg/dl | 121.15 mg/dl | −26.35 mg/dl | −8.1% | −14.4% |
| niacin (n = 34) | | | | | |

Administration of A-002 alone resulted in 3.7% increase in TG versus baseline and a 2.6% decrease in TG versus placebo, while administration of niacin drugs alone resulted in a 9.7% increase in TG versus baseline and a 3.4% increase in TG versus placebo. If A-002 and niacin drugs functioned in a simple additive manner, administration of a combination of A-002 and niacin drugs would be expected to increase TG levels by 13.4% versus baseline (3.7% plus 9.7%) and by 0.8% versus placebo (−2.6% plus 3.4%). However, the combined effect of A-002 and niacin drug administration was instead an 8.1% decrease in TG levels versus baseline and a 14.4% decrease in TG levels versus placebo, indicating that A-002 and niacin drugs unexpectedly decrease TG levels in a synergistic manner.

### Example 2

### A-002/Niacin Formulations

Fixed dose tablets containing A-002 and/or one or more niacin drugs may be generated using methods known in the art.

Previous studies have established the feasibility of formulating A-002 into a tablet comprising the ingredients set forth in Table 3. One of ordinary skill in the art will recognize that additional components may be added to this formulation. For example, a compound such as calcium carbonate may be added to the formulation to enhance dissolution and solubility. Likewise, one of skill in the art will recognize that this formulation is just one example of a generic A-002 formulation, and that the identity and weight of the recited components within the formulation may be varied without undue experimentation.

TABLE 3
Generic A-002 formulation

| | Typical weight |
|---|---|
| Component | percentage |
| A-002 | Varies depending on |
| | desired dosage |
| Anhydrous lactose | 20-50% |
| Lactose monohydrate | 20-50% |
| Hydroxypropyl cellulose | 2-6% |
| Croscarmellose sodium | 0.5-5% |
| Polysorbate 80 | 0.1-3% |
| Microcrystalline cellulose | 5-20% |
| Magnesium stearate | 0.25-3% |

A combination drug containing A-002 and one or more niacin drugs may be formulated by incorporating one or more niacin drugs into a formulation similar to that set forth in Table 4. Niacin drugs are routinely formulated as extended release tablets comprising a fixed dosage of niacin in combination with inactive ingredients such as hydroxypropyl methylcellulose, povidone, steric acid, polyvinyl pyrollidone, and polyethylene glycol. Therefore, a combined A-002/niacin formulation may contain one or more of these inactive ingredients in lieu of or in addition to any of the ingredients set forth in Table 3.

A-002 and niacin may be formulated into a tablet, capsule, implantable wafer or disc, or other form where active ingredients are released immediately, or they may be formulated for extended or delayed release using commonly available excipients and technology. Extended release formulations may comprise a plurality of particles or beads with a distintegratable coating, wherein one or more active ingredients are incorporated into or distributed along the surface of each particle or bead. A single formulation may contain particles or beads with a variety of coating thicknesses, such that the active ingredient(s) are released at different times following administration. Such formulations may result in substantially constant blood levels of the active ingredient(s) over an extended period. Alternatively, these formulations may result in a pulsed plasma profile, wherein the active ingredient(s) are released in cycles. Dosage strengths should encompass the range of 500-1000 mg for niacin and 250-500 mg for A-002.

Triple combination products may be formulated that comprise A-002, one or more niacin drugs, and one or more statins such as atorvastatin, cerivastatin, fluvastatin lovastatin, mevastatin, pitavastatin, pravastatin, rosuvastatin, or simvastatin. Commonly available excipients that are routinely used for the formulation of statins into solid oral dosage forms may be incorporated into these formulations.

As stated above, the foregoing is merely intended to illustrate various embodiments of the present invention. The specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein. All references cited herein are incorporated by reference as if fully set forth herein.

### REFERENCES

1. Assmann et al. 1998. Eur Heart J 19 (Suppl A):A2-A11.
2. Benyo et al. 2005. J Clin Invest 115:3634.
3. Cheng et al. 2006. Proc Natl Acad Sci USA 103:6682.
4. Gordon et al. 1989. Circulation 79:8-15.
5. Kuvin et al. 2006. Am J Cardiol 98:743.
6. Offermanns et al. 2006. Trends Pharmacol Sci 27:384.
7. Papaliodis et al. 2008. J Pharmacol Exper Ther 327:665.
8. Pike et al. 2005. J Clin Invest 115-3400.
9. Richman et al. 2007. J Biol Chem 282:18028.

What is claimed is:

1. A method of increasing HDL levels in a subject in need thereof comprising administering a therapeutically effective amount of one or more sPLA₂ inhibitors and a therapeutically effective amount of one or more niacin drugs.

2. The method of claim 1, wherein said one or more sPLA₂ inhibitors comprise 3-(2-Amino-1,2-dioxoethyl)-2-ethyl-1-(phenylmethyl)-1H-indol-4-yl)oxy)acetic acid or a pharmaceutically acceptable prodrug, salt, solvate, polymorph, or co-crystal thereof.

3. The method of claim 2, wherein said prodrug is selected from the group consisting of a C₁-C₆ alkyl ester prodrug, an acyloxyalkyl ester prodrug, and an alkyloxycarbonyloxyalkyl ester prodrug.

4. The method of claim 3, wherein said C₁-C₆ alkyl ester is [[3-(2-Amino-1,2-dioxoethyl)-2-ethyl-1-(phenylmethyl)-1H-indol-4-yl]oxy]acetic acid methyl ester.

5. The method of claim 1, wherein said niacin drug is selected from the group consisting of niacin, nicotinic acid, acipimox, immediate release niacin, extended release niacin, extended release niacin in combination with lovastatin, extended release niacin in combination with simvastatin, extended release niacin in combination with laropiprant, xanthinol niacinate, 1-methylnicotinamide, and 1-methyl-N′-hydroxymethylnicotinamide.

6. The method of claim 1, further comprising administering a therapeutically effective amount of one or more statins.

7. A method of decreasing TG levels in a subject in need thereof comprising administering a therapeutically effective amount of one or more sPLA₂ inhibitors and a therapeutically effective amount of one or more niacin drugs.

8. The method of claim 7, wherein said one or more sPLA₂ inhibitors comprise 3-(2-Amino-1,2-dioxoethyl)-2-ethyl-1-(phenylmethyl)-1H-indol-4-yl)oxy)acetic acid or a pharmaceutically acceptable prodrug, salt, solvate, polymorph, or co-crystal thereof.

9. The method of claim 8, wherein said prodrug is selected from the group consisting of a C₁-C₆ alkyl ester prodrug, an acyloxyalkyl ester prodrug, and an alkyloxycarbonyloxyalkyl ester prodrug.

10. The method of claim 9, wherein said C₁-C₆ alkyl ester is [[3-(2-Amino-1,2-dioxoethyl)-2-ethyl-1-(phenylmethyl)-1H-indol-4-yl]oxy]acetic acid methyl ester.

11. The method of claim 7, wherein said niacin drug is selected from the group consisting of niacin, nicotinic acid, acipimox, immediate release niacin, extended release niacin, extended release niacin in combination with lovastatin, extended release niacin in combination with simvastatin, extended release niacin in combination with laropiprant, xanthinol niacinate, 1-methylnicotinamide, and 1-methyl-N′-hydroxymethylnicotinamide.

12. The method of claim 7, further comprising administering a therapeutically effective amount of one or more statins.

13. A method of increasing the HDL/LDL ratio in a subject in need thereof comprising administering a therapeutically effective amount of one or more sPLA₂ inhibitors and a therapeutically effective amount of one or more niacin drugs.

14. The method of claim 13, wherein said one or more sPLA₂ inhibitors comprise 3-(2-Amino-1,2-dioxoethyl)-2-ethyl-1-(phenylmethyl)-1H-indol-4-yl)oxy)acetic acid or a pharmaceutically acceptable prodrug, salt, solvate, polymorph, or co-crystal thereof.

15. The method of claim 14, wherein said prodrug is selected from the group consisting of a C₁-C₆ alkyl ester prodrug, an acyloxyalkyl ester prodrug, and an alkyloxycarbonyloxyalkyl ester prodrug.

16. The method of claim 15, wherein said C₁-C₆ alkyl ester is [[3-(2-Amino-1,2-dioxoethyl)-2-ethyl-1-(phenylmethyl)-1H-indol-4-yl]oxy]acetic acid methyl ester.

17. The method of claim 13, wherein said niacin drug is selected from the group consisting of niacin, nicotinic acid, acipimox, immediate release niacin, extended release niacin, extended release niacin in combination with lovastatin, extended release niacin in combination with simvastatin, extended release niacin in combination with laropiprant, xanthinol niacinate, 1-methylnicotinamide, and 1-methyl-N′-hydroxymethylnicotinamide.

18. The method of claim 13, further comprising administering a therapeutically effective amount of one or more statins.

19. A method of treating cardiovascular disease or a condition associated with cardiovascular disease in a subject in need thereof comprising administering a therapeutically effective amount of one or more sPLA₂ inhibitors and a therapeutically effective amount of one or more niacin drugs.

20. The method of claim 19, wherein said one or more sPLA₂ inhibitors comprise 3-(2-Amino-1,2-dioxoethyl)-2-ethyl-1-(phenylmethyl)-1H-indol-4-yl)oxy)acetic acid or a pharmaceutically acceptable prodrug, salt, solvate, polymorph, or co-crystal thereof.

21. The method of claim 20, wherein said prodrug is selected from the group consisting of a C₁-C₆ alkyl ester prodrug, an acyloxyalkyl ester prodrug, and an alkyloxycarbonyloxyalkyl ester prodrug.

22. The method of claim 21, wherein said C₁-C₆ alkyl ester is [[3-(2-Amino-1,2-dioxoethyl)-2-ethyl-1-(phenylmethyl)-1H-indol-4-yl]oxy]acetic acid methyl ester.

23. The method of claim 19, wherein said niacin drug is selected from the group consisting of niacin, nicotinic acid, acipimox, immediate release niacin, extended release niacin, extended release niacin in combination with lovastatin, extended release niacin in combination with simvastatin, extended release niacin in combination with laropiprant, xanthinol niacinate, 1-methylnicotinamide, and 1-methyl-N′-hydroxymethylnicotinamide.

24. The method of claim 19, further comprising administering a therapeutically effective amount of one or more statins.

25. The method of claim 19, wherein said cardiovascular disease is selected from the group consisting of coronary artery disease and coronary heart disease.

26. A composition comprising one or more sPLA₂ inhibitors and one or more niacin drugs.

27. The composition of claim 26, wherein said one or more sPLA₂ inhibitors comprise 3-(2-Amino-1,2-dioxoethyl)-2-ethyl-1-(phenylmethyl)-1H-indol-4-yl)oxy)acetic acid or a pharmaceutically acceptable prodrug, salt, or solvate thereof.

28. The composition of claim 27, wherein said prodrug is selected from the group consisting of a C₁-C₆ alkyl ester prodrug, an acyloxyalkyl ester prodrug, and an alkyloxycarbonyloxyalkyl ester prodrug.

29. The composition of claim 28, wherein said C₁-C₆ alkyl ester prodrug is [[3-(2-Amino-1,2-dioxoethyl)-2-ethyl-1-(phenylmethyl)-1H-indol-4-yl]oxy]acetic acid methyl ester.

30. The composition of claim 26, wherein said niacin drug is selected from the group consisting of niacin, nicotinic acid, acipimox, immediate release niacin, extended release niacin, extended release niacin in combination with lovastatin, extended release niacin in combination with simvastatin, extended release niacin in combination with laropiprant, xanthinol niacinate, 1-methylnicotinamide, and 1-methyl-N′-hydroxymethylnicotinamide.

31. A method of increasing the effectiveness of niacin drug administration in a subject in need thereof comprising administering a therapeutically effective amount of one or more sPLA₂ inhibitors.

32. A kit comprising one or more sPLA₂ inhibitors and one or more niacin drugs.
